(19) United States
(12) Patent Application Publication
(10) Pub. No.: US 2025/0041245 A1
(43) Pub. Date: Feb. 6, 2025

(54) ENDOXIFEN FOR TREATMENT OF CANCERS

(71) Applicant: Atossa Therapeutics, Inc., Seattle, WA (US)

(72) Inventors: Steven C. QUAY, Seattle, WA (US); Carla GRANDORI, Seattle, WA (US); Katannya KAPELI, Kaneohe, HI (US)

(21) Appl. No.: 18/704,892

(22) PCT Filed: Oct. 27, 2022
(86) PCT No.: PCT/US2022/048061

§ 371 Date: Apr. 25, 2024

Related U.S. Application Data
(60) Provisional application No. 63/272,869, filed on Oct. 28, 2021.

(51) Int. Cl. A61K 31/138; A61K 45/06; A61P 35/04

(52) U.S. Cl. CPC A61K 31/138; A61K 45/06; A61P 35/04

(57) ABSTRACT

Described herein are endoxifen compositions and methods using endoxifen compositions for treating various cancers. The endoxifen compositions may be used to treat cancers such as skin cancers, gastrointestinal cancers, neuroblastoma, breast cancers, cervical cancers, or ovarian cancers. A method of treating various cancers may include administering endoxifen to a subject having skin cancer, gastrointestinal cancer, neuroblastoma, breast cancer, cervical cancer, or ovarian cancer.

### CROSS-REFERENCE

The present application claims the benefit of U.S. Provisional Application No. 63/272,869, entitled “ENDOXIFEN FOR TREATMENT OF CANCERS,” filed on Oct. 28, 2021, which application is herein incorporated by reference in its entirety for all purposes.

### BACKGROUND

While cancer survival rates have been increasing over the last few decades, cancer remains the second leading cause of death in the United States. Despite extensive research into treatments and prevention, drug resistance and relapse remain limiting factors in developing cancer cures. To combat these and other factors, there is a need for cancer therapies capable of overcoming drug resistance and preventing relapse.

### SUMMARY

In various aspects, the present disclosure provides a method of treating a gastrointestinal cancer in a subject in need thereof, the method comprising administering to the subject a therapeutically effective amount of endoxifen, thereby treating the gastrointestinal cancer.

In some aspects, the gastrointestinal cancer is a colorectal cancer, a gastric cancer, a pancreatic cancer, an esophageal cancer, a rectal cancer, a biliary cancer, a cholangiocarcinoma, or a combination thereof. In some aspects, the gastrointestinal cancer is a carcinoma. In some aspects, the gastrointestinal cancer is an adenocarcinoma. In some aspects, the gastrointestinal cancer is a colorectal cancer. In some aspects, the gastrointestinal cancer is a colon cancer. In some aspects, the colon cancer is sigmoid colon cancer, adenocarcinoma of the colon, stage III colorectal cancer, stage IV colorectal cancer, or descending colon cancer. In some aspects, the colorectal cancer is colon cancer, sigmoid colon cancer, adenocarcinoma of the colon, stage III colorectal cancer, stage IV colorectal cancer, or descending colon cancer.

In some aspects, the gastrointestinal cancer is a gastric cancer. In some aspects, the gastrointestinal cancer is a pancreatic cancer. In some aspects, the pancreatic cancer is pancreatic ductal adenocarcinoma, metastatic pancreatic ductal adenocarcinoma, or stage III metastatic pancreatic ductal adenocarcinoma.

In some aspects, the gastrointestinal cancer is an esophageal cancer. In some aspects, the esophageal cancer is esophageal adenocarcinoma or metastatic esophageal cancer. In some aspects, the gastrointestinal cancer is a rectal cancer. In some aspects, the rectal cancer is rectum adenocarcinoma. In some aspects, the gastrointestinal cancer is a cholangiocarcinoma. In some aspects, the cholangiocarcinoma is metastatic cholangiocarcinoma. In some aspects, the gastrointestinal cancer is metastatic. In some aspects, the gastrointestinal cancer is recurrent.

In various aspects, the present disclosure provides a method of treating a skin cancer in a subject in need thereof, the method comprising administering to the subject a therapeutically effective amount of endoxifen, thereby treating the skin cancer.

In some aspects, the skin cancer is a melanoma. In some aspects, the melanoma is acral melanoma, recurrent acral melanoma, or anorectal melanoma. In some aspects, the skin cancer is metastatic. In some aspects, the skin cancer is recurrent.

In various aspects, the present disclosure provides a method of treating neuroblastoma in a subject in need thereof, the method comprising administering to the subject a therapeutically effective amount of endoxifen, thereby treating the neuroblastoma.

In some aspects, the neuroblastoma is metastatic. In some aspects, the neuroblastoma is recurrent.

In some aspects, the endoxifen is (Z)-endoxifen. In some aspects, the (Z)-endoxifen has an isomeric purity of at least 90%. In some aspects, the endoxifen administered orally, topically, rectally, intravenously, intra-arterially, parenterally, transdermally, or via inhalation. In some aspects, the endoxifen is administered orally. In some aspects, the endoxifen is formulated as a sustained-release composition or a delayed release composition. In some aspects, the endoxifen is formulated as a capsule or a tablet. In some aspects, the endoxifen is administered topically or transdermally. In some aspects, the endoxifen is formulated is a cream, a gel, a cream, an emulsion, a lotion, an ointment, a solution, a paste, a patch, or an oil.

In some aspects, the method comprises administering the endoxifen to the subject daily. In some aspects, the method comprises administering the endoxifen to the subject at a dose of no less than 1 mg and no more than 160 mg per day. In some aspects, the method comprises administering the endoxifen to the subject at a dose of no less than 1 mg and no more than 40 mg per day. In some aspects, the method comprises administering the endoxifen to the subject at a dose of no less than 1 mg and no more than 10 mg per day. In some aspects, the method comprises administering the endoxifen to the subject at a dose of no less than 2 mg and no more than 5 mg per day. In some aspects, the method comprises administering the endoxifen 1, 2, 3, or 4 times per day. In some aspects, the method comprises administering the endoxifen for at least 7, at least 14, at least 21, or at least 28 days.

In some aspects, the administering achieves a systemic endoxifen Cₘₐₓ of between about 0.01 and about 50 μM in the subject. In some aspects, the administering achieves a systemic endoxifen Cₘₐₓ of between about 1 and about 10 μM in the subject. In some aspects, the administering maintains a systemic endoxifen concentration of at least 1 μM for at least one week in the subject

In some aspects, the method further comprises administering an additional therapeutic agent. In some aspects, the additional therapeutic agent comprises an anti-cancer agent. In some aspects, the anti-cancer agent is selected from the group consisting of bicalutamide, enzalutamide, trastuzumab, atezolizumab, alpelisib, olaparib, talazoparib, ribociclib, neratinib, an antineoplastic, capecitabine, carboplatin, cisplatin, cyclophosphamide, docetaxel, doxorubicin, pegylated liposomal doxorubicin, epirubicin, fluorouracil, gemcitabine, methotrexate, paclitaxel, protein-bound paclitaxel, vinorelbine, eribulin, ixabepilone, an immune checkpoint inhibitor, a PD1 inhibitor, a PD-L1 inhibitor, a CTLA4 inhibitor, and an ATP-cassette binding protein inhibitor. In some aspects, the additional therapeutic agent comprises a selective serotonin reuptake inhibitor. In some aspects, the selective serotonin reuptake inhibitor comprises citalopram, escitalopram, fluoxetine, paroxetine, sertraline, or vilazodone.

In some aspects, the method further comprises surgical tumor removal. In some aspects, the administering the therapeutically effective amount of endoxifen is concurrent with, subsequent to, or concurrent with and subsequent to the surgical tumor removal. In some aspects, the method further comprises radiotherapy. In some aspects, the administering the therapeutically effective amount of endoxifen is concurrent with, subsequent to, or concurrent with and subsequent to the radiotherapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a histogram depicting the number of samples derived from patients diagnosed with various cancer types, including Breast Cancer (BRST), Cervical Carcinoma (CC), Cholangiocarcinoma (CHOL), Colorectal Cancer (CRC), Esophageal Adenocarcinoma (EAC), Endometrial Carcinoma (EC), Gastric Cancer (GASC), Leiomyosarcoma (LYMSC), Myxofibrosarcoma (MFSC), Melanoma (MHLNM), Neuroblastoma (NBL), Ovarian Cancer (OVA), Pancreatic Ductal Adenocarcinoma (PDAC), Primary Peritoneal Carcinoma (PPC), Rectal Cancer (RC), Sarcoma (SC), and Thyroid Cancer (THYRD), screened for endoxifen response using a personalized cancer treatment screening assay. Shading of the histogram bars represents the response category of individual patient samples to endoxifen, as determined by the personalized cancer treatment screening assay.

FIG. 2A, FIG. 2B, FIG. 2C, and FIG. 2D show endoxifen dose-response curves for individual patients diagnosed with breast cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 3A, FIG. 3B, FIG. 3C, and FIG. 3D show additional endoxifen dose-response curves for individual patients diagnosed with breast cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 4A, FIG. 4B, FIG. 4C, and FIG. 4D show additional endoxifen dose-response curves for individual patients diagnosed with breast cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 5A, FIG. 5B, and FIG. 5C show additional endoxifen dose-response curves for individual patients diagnosed with breast cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 6 shows an endoxifen dose-response curve for a patient diagnosed with cervical cancer. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 7A, FIG. 7B, and FIG. 7C show endoxifen dose-response curves for individual patients diagnosed with cholangiocarcinoma. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 8A, FIG. 8B, and FIG. 8C show endoxifen dose-response curves for individual patients diagnosed with colorectal cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 9A, FIG. 9B, FIG. 9C, and FIG. 9D show additional endoxifen dose-response curves for individual patients diagnosed with colorectal cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 10A, FIG. 10B, FIG. 10C, and FIG. 10D show additional endoxifen dose-response curves for individual patients diagnosed with colorectal cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 11A, FIG. 11B, FIG. 11C, and FIG. 11D show additional endoxifen dose-response curves for individual patients diagnosed with colorectal cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 12 shows an endoxifen dose-response curves for a patient diagnosed with esophageal cancer. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 13 shows an endoxifen dose-response curve for a patient diagnosed with endometrial cancer. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 14A and FIG. 14B show endoxifen dose-response curves for individual patients diagnosed with gastric cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 15A, FIG. 15B, FIG. 15C, and FIG. 15D show additional endoxifen dose-response curves for individual patients diagnosed with gastric cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 16A, FIG. 16B, and FIG. 16C show endoxifen dose-response curves for individual patients diagnosed with leiomyosarcoma. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 17 shows an endoxifen dose-response curve for a patient diagnosed with myxofibrosarcoma. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 18A, FIG. 18B, FIG. 18C, and FIG. 18D show endoxifen dose-response curves for individual patients diagnosed with melanoma. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 19A, FIG. 19B, and FIG. 19C show endoxifen dose-response curves for individual patients diagnosed with neuroblastoma. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 20A, FIG. 20B, and FIG. 20C show endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 21A, FIG. 21B, FIG. 21C, and FIG. 21D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 22A, FIG. 22B, FIG. 22C, and FIG. 22D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 23A, FIG. 23B, FIG. 23C, and FIG. 23D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 24A, FIG. 24B, FIG. 24C, and FIG. 24D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 25A, FIG. 25B, FIG. 25C, and FIG. 25D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 26A, FIG. 26B, FIG. 26C, and FIG. 26D show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 27A and FIG. 27B show additional endoxifen dose-response curves for individual patients diagnosed with ovarian cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 28A, FIG. 28B, and FIG. 28C show endoxifen dose-response curves for individual patients diagnosed with pancreatic cancer. Endoxifen dose-response was measured using patient-derived tumor organoids (PDTOs) derived from the individual patients. Response parameters, including the SEngine Precision Medicine (SPM) score, IC₅₀, area under the curve (AUC), and goodness of fit (GOF) of the response curve, are shown in the inset.

FIG. 29 shows an endoxifen dose-response curves for a patient diagnosed with primary peritoneal carcinoma. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 30 shows an endoxifen dose-response curve for a patient diagnosed with rectal cancer. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 31 shows an endoxifen dose-response curve for a patient diagnosed with sarcoma. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 32 shows an endoxifen dose-response curve for a patient diagnosed with thyroid cancer. Endoxifen dose-response was measured using patient-derived tumor organoids (PDTOs) derived from the patient. Response parameters, including the SEngine Precision Medicine (SPM) score, IC₅₀, area under the curve (AUC), and goodness of fit (GOF) of the response curve, are shown in the inset.

FIG. 33 shows a histogram depicting the distribution of cancer types, including Breast Cancer (BRST), Cervical Carcinoma (CC), Cholangiocarcinoma (CHOL), Colorectal Cancer (CRC), Esophageal Adenocarcinoma (EAC), Endometrial Carcinoma (EC), Gastric Cancer (GASC), Leiomyosarcoma (LYMSC), Myxofibrosarcoma (MFSC), Melanoma (MLNM), Neuroblastoma (NBL), Ovarian Cancer (OVA), Pancreatic Ductal Adenocarcinoma (PDAC), Primary Peritoneal Carcinoma (PPC), Rectal Cancer (RC), Sarcoma (SC), and Thyroid Cancer (THYRD), screened for endoxifen response using a personalized cancer treatment screening assay.

FIG. 34A is an XRPD pattern obtained from a sample of Form I of endoxifen.

FIG. 34B is an XRPD pattern obtained from a sample of Form II of endoxifen.

FIG. 34C is an XRPD pattern obtained from a sample of Form III of endoxifen.

### DETAILED DESCRIPTION

Efficacy of cancer therapies is highly dependent on cancer type such that drugs that are highly effective in one cancer type may have limited efficacy in other cancers. Often, this results from drugs that target specific pathways disrupted or upregulated in one cancer type but not in others. The present disclosure provides composition and methods for treating various cancers using endoxifen. Endoxifen is an active metabolite of tamoxifen. In the liver, is broken down into active compounds, or metabolites. One of the active tamoxifen metabolites is endoxifen, also referred to as 4-hydroxy-N-desmethyltamoxifen. Endoxifen (e.g., Z-endoxifen) is a selective estrogen receptor modulator (SERM) that functions as a competitive partial agonist of estrogen receptor in a tissue-specific manner. Z-endoxifen has robust antitumor and anti-estrogenic activity compared to tamoxifen therapy and aromatase inhibitor therapy. Endoxifen may be beneficial in treating cancers in patients that are resistant to other hormone therapies, such as tamoxifen, aromatase inhibitors, or fulvestrant, in part because endoxifen functions independently of metabolic enzymes such as CYP2D6.

Described herein are compositions comprising endoxifen (e.g., Z-endoxifen) that may be used for treating various cancers (e.g., melanoma, colorectal cancer, gastric cancer, neuroblastoma, pancreatic cancer, esophageal cancer, rectal cancer, cholangiocarcinoma, breast cancer, cervical cancer, or ovarian cancer). Also described herein are methods of treating various cancers (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) using endoxifen compositions. A method of treating various cancers may comprise administering an endoxifen composition to a subject. The endoxifen composition may comprise Z-endoxifen, and polymorphs and salts thereof. In some embodiments, the endoxifen composition may be used in a method of treating melanoma, colorectal cancer, gastric cancer, neuroblastoma, pancreatic cancer, esophageal cancer, rectal cancer, cholangiocarcinoma, breast cancer, cervical cancer, or ovarian cancer. For example, the endoxifen composition may be used to treat melanoma, colorectal cancer, gastric cancer, neuroblastoma, pancreatic cancer, esophageal cancer, rectal cancer, or cholangiocarcinoma. In some embodiments, the endoxifen composition may be used to treat triple negative breast cancer (e.g., breast cancer that lacks or has low levels of estrogen receptor, progesterone receptor, and HER2). In some embodiments, the endoxifen compositions described herein may be used to treat a gastrointestinal cancer, such as colorectal cancer, gastric cancer, pancreatic cancer, esophageal cancer, rectal cancer, cholangiocarcinoma. In some embodiments, the endoxifen compositions described herein may be used to treat a skin cancer, such as melanoma. In some embodiments, the endoxifen compositions described herein may be used to treat a neuroblastoma.

The compositions and methods described herein may be used in combination with other therapies (e.g., a chemotherapy, a drug, a radiation therapy, or surgery) to treat the cancer or associated symptoms (e.g., pain, depression, anxiety, nausea, fatigue, or loss of appetite). For example, an endoxifen composition may be administered in combination with an antidepressant (e.g., a serotonin reuptake inhibitor). In another example, an endoxifen composition may be administered before, after, or concurrent with surgery to remove the cancer or cancerous tissue. In another example, the endoxifen composition may be administered in combination with an additional anti-cancer agent. The anti-cancer agent may be formulated with the endoxifen in a pharmaceutical composition or administered separately. In some embodiments, an anti-cancer agent for use in combination with endoxifen to treat a cancer may be identified using a drug screen. For example, a drug combination comprising endoxifen that is effective at treating a cancer (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) may be identified using a patient-derived tumor organoid (PDTO) screen.

### Methods of Treating Cancers

A method of treating various cancers may comprise administering to a subject a therapy comprising endoxifen (e.g., Z-endoxifen). In some embodiments, the endoxifen is administered orally. In some embodiments, the endoxifen is administered topically. In some embodiments, the endoxifen is administered orally, topically, transdermally, rectally, intravenously, intra-arterially, intra-ovarianly, vaginally, parenterally, or via inhalation. In some embodiments, the endoxifen is formulated as a sustained release composition. A composition comprising endoxifen may be administered 1, 2, 3, 4, 5, 6, 7, 8, 9, or 10 times per day. In some embodiments, a composition comprising endoxifen may be administered 1, 2, 3, 4, 5, 6, 7, 8, 9, or 10 times per day. Treatment may be administered for at least 7, 14, 21, 28, 30, 35, 42, 49, 56, or 60 days. In some embodiments, treatment may be administered until a cancer is treated (e.g., by eliminating or reducing the size of a tumor). In some embodiments, treatment may be administered until unacceptable toxicity is observed in the subject. In some embodiments, endoxifen (e.g., Z-endoxifen) may be administered to treat a cancer at a dose of from about 1 mg to about 160 mg, from about 1 mg to about 40 mg, from about 1 mg to about 10 mg, or from about 2 mg to about 5 mg per day.

In some embodiments, a method of treating a cancer may comprise administering endoxifen in combination with an additional therapeutic agent. For example, a method of treating a cancer may comprise administering endoxifen and an additional anti-cancer agent. In some embodiments, the anti-cancer agent may be bicalutamide, enzalutamide, trastuzumab, atezolizumab, alpelisib, olaparib, talazoparib, ribociclib, neratinib, an antineoplastic, capecitabine, carboplatin, cisplatin, cyclophosphamide, docetaxel, doxorubicin, pegylated liposomal doxorubicin, epirubicin, fluorouracil, gemcitabine, methotrexate, paclitaxel, protein-bound paclitaxel, vinorelbine, eribulin, ixabepilone, an immune checkpoint inhibitor, a PD1 inhibitor, a PD-L1 inhibitor, a CTLA4 inhibitor, or an ATP-cassette binding protein inhibitor. In some embodiments, a method of treating a cancer may comprise administering endoxifen and a selective serotonin reuptake inhibitor (e.g., citalopram, escitalopram, fluoxetine, paroxetine, sertraline, or vilazodone).

In some embodiments, a dosage of endoxifen for treating a certain cancer type may be selected based on an IC₅₀ of endoxifen determined in vitro for the cancer type of interest. The IC₅₀ may be determined as the concentration of endoxifen that inhibits 50% of the tumor cells of that cancer type in vitro. For example, a dosage of endoxifen may be selected to treat a cancer type such that an in vivo concentration is achieved within the subject (e.g., a maximum serum concentration (Cₘₐₓ)) that is one tenth the IC₅₀, at least one fifth the IC₅₀, at least one third the IC₅₀, at least half IC₅₀, or at least the IC₅₀. The IC₅₀ of endoxifen for the specific cancer type (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) may be determined by generating a dose-response curve using patient-derived tumor organoids (PDTOs). In some embodiments, the IC₅₀ may be patient specific, cancer type specific, or both. In some embodiments, the dosage for treating a subject may be adjusted based on an IC₅₀ measured in tumor organoids derived from the subject.

### (i) Methods of Treating Gastrointestinal Cancers

A surprising discovery disclosed herein is that endoxifen can be efficacious for treating gastrointestinal cancers, which herein can refer to cancers which originate in the gastrointestinal (GI) tract or an accessory organ of digestion. Gastrointestinal cancers encompass a broad range cell malignancies, including cancers of the pharynx, esophagus, stomach, biliary system, pancreas, small intestine, large intestine, rectum, anus, spleen, liver, gallbladder, peritoneum, and certain cells and tissues associated therewith. While endoxifen would not be anticipated to treat malignancies of some gastrointestinal tissues, for example those in which estrogen receptor β agonism affects apparent tumor suppression (Y. Niv. Eur J Gastroenterol Hepatol, 2015; 27(12):1438-42) or those with low estrogen receptor and estrogen receptor complex protein distributions, organoid studies (e.g., those outlined in EXAMPLES 5-8 and 17-19) identified that endoxifen can exhibit pronounced and generalizable cancer cell clearance from gastrointestinal tissues. Furthermore, the organoid studies disclosed herein indicate that endoxifen effectively treats gastrointestinal cancers in the absence of immune-mediated apoptosis and clearance, suggesting that endoxifen may be ideal for treating a number of recalcitrant and metastatic gastrointestinal cancers, for example those which are immune suppressive, anoikis resistant, or have a high incidence of circulating tumor cell (CTC) formation.

Leveraging these observations, aspects of the present disclosure provide methods of treating gastrointestinal cancer comprising administering a therapeutically effective amount of endoxifen to a subject in need thereof. In some cases, the gastrointestinal cancer is an esophageal cancer, a gastric cancer, a cancer of the small intestine, a cancer of the appendix, a colorectal cancer, an anal cancer, a liver cancer, a biliary cancer (e.g., a cholangiocarcinoma), a pancreatic cancer, a cancer of the peritoneum, or a combination thereof. In some cases, the gastrointestinal cancer is a colorectal cancer, a gastric cancer, a pancreatic cancer, an esophageal cancer, a rectal cancer, a biliary cancer, a cholangiocarcinoma, or a combination thereof. In some cases, the gastrointestinal cancer is metastatic. In some cases, the gastrointestinal cancer is recurrent.

In some cases, the gastrointestinal cancer is a carcinoma. In some cases, the gastrointestinal cancer is an adenocarcinoma. In some cases, the adenocarcinoma is an adenocarcinoma of the colon, a pancreatic ductal adenocarcinoma, a rectal adenocarcinoma, or a combination thereof. In some cases, the carcinoma is metastatic (e.g., metastatic adenocarcinoma). In some cases, the carcinoma is recurrent.

In some cases, the gastrointestinal cancer is a colorectal cancer. In some cases, the colorectal cancer is a cancer of the caecum, a cancer of the ascending colon, a cancer of the transverse colon, a cancer of the descending colon, a cancer of the sigmoid colon, a cancer of the rectum, or a combination thereof. In some cases, the colorectal cancer is a colon cancer. In some cases, the colon cancer is a sigmoid colon cancer, an adenocarcinoma of the colon, a descending colon cancer, or a combination of cancers thereof. In some cases, the colorectal cancer is metastatic. In some cases, the colorectal cancer is recurrent.

In some cases, the gastrointestinal cancer is a gastric cancer. In some cases, the gastric cancer is gastric carcinoma, gastric adenocarcinoma, gastrointestinal stromal cancer, signet ring cell carcinoma, gastric lymphoma, linitis plastica, hereditary diffuse gastric cancer, mucosa-associated lymphoid tissue, or a combination thereof. In some cases, the gastrointestinal cancer is an adenocarcinoma. In some cases, the gastric cancer is metastatic. In some cases, the gastric cancer is recurrent.

In some cases, the gastrointestinal cancer is a pancreatic cancer. In some cases, the pancreatic cancer is an exocrine pancreatic cancer, a cystic neoplasm, a serous microcystic adenoma, a pancreatoblastoma, an intraductal papillary mucinous neoplasm, a pseudopapillary neoplasm, a mucinous cystic neoplasm, or a combination thereof. In some cases, the pancreatic cancer is an exocrine pancreatic cancer. In some cases, the pancreatic cancer is a pancreatic adenocarcinoma. In some cases, the adenocarcinoma is ductal pancreatic adenocarcinoma. In some cases, the adenocarcinoma is metastatic pancreatic ductal adenocarcinoma. In some cases, the pancreatic cancer is metastatic. In some cases, the pancreatic cancer is recurrent.

### (ii) Methods of Treating Skin Cancers

Aspects of the present disclosure provide methods of treating skin cancer comprising administering a therapeutically effective amount of endoxifen to a subject in need thereof. As skin cancers can diminish estrogen receptor alpha expression, skin cancers can be poorly responsive to selective estrogen receptor modulators such as endoxifen. Nonetheless, it was hypothesized herein that endoxifen may exhibit activity against some forms of skin cancer. This hypothesis was confirmed by skin cancer organoid assays (e.g., as outlined in EXAMPLE 20), which identified endoxifen IC₅₀ values of less than 10 μM for multiple skin cancers. As the organoids were substantially free of immune cells, these assays further demonstrated that endoxifen activity against skin cancers can be independent of immune inhibition and cancer cell clearance.

In some cases, the skin cancer is a carcinoma, a melanoma, a cutaneous T-cell lymphoma, a dermatofibrosarcoma, or a combination thereof. In some cases, the skin cancer is a carcinoma. In some cases, the carcinoma is selected from the group consisting of cutaneous T-cell lymphoma, dermatofibrosarcoma protuberans, Merkel cell carcinoma, sebaceous carcinoma, basal cell carcinoma, squamous cell carcinoma, or a combination thereof. In some cases, the skin cancer is a cutaneous T-cell lymphoma. In some cases, the skin cancer is a dermatofibrosarcoma. In some cases, the skin cancer is metastatic. In some cases, the skin cancer is recurrent.

In some cases, the skin cancer is a melanoma. In some cases, the melanoma is a superficial spreading melanoma, a nodular melanoma, a lentigo maligna melanoma, an acral melanoma, a mucosal melanoma, a desmoplastic melanoma, a uveal melanoma, a vaginal melanoma, a polypoid melanoma, or a combination thereof. In some cases, the melanoma is an acral melanoma, a recurrent acral melanoma, an anorectal melanoma, or a combination thereof. In some cases, the melanoma is an acral melanoma. In some cases, the acral melanoma is a recurrent acral melanoma. In some cases, the melanoma is an anorectal melanoma. In some cases, the melanoma is a recurrent melanoma. In some cases, the melanoma is metastatic.

(iii) Methods of Treating Neuroblastomas

Aspects of the present disclosure provide methods of treating neuroblastoma comprising administering a therapeutically effective amount of endoxifen to a subject in need thereof. Neuroblastomas typically manifest from neuroblasts within sympathetic nervous tissue, and can therefore arise in range of locations including the neck, chest, and spinal column. Neuroblastomas often exhibit high degrees of cellular heterogeneity, and can therefore be difficult to treat when in advanced stages and following dissemination. As disclosed herein (for example in EXAMPLE 10), endoxifen exhibits a low IC₅₀ for some neuroblastomas, and can therefore provide an effective means for neuroblastoma treatment as well as remission maintenance. For some methods of treating neuroblastoma disclosed herein, the neuroblastoma is metastatic. For some methods of treating neuroblastoma disclosed herein, the neuroblastoma is recurrent.

### (iv) Cancer Treatment Dosing

In some embodiments, a dosage of endoxifen for treating melanoma (e.g., acral melanoma, recurrent acral melanoma, or anorectal melanoma) may be selected to achieve a Cₘₐₓ (e.g., a systemic Cₘₐₓ) in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating melanoma (e.g., acral melanoma, recurrent acral melanoma, or anorectal melanoma) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM.

In some embodiments, a dosage of endoxifen for treating colorectal cancer is selected to achieve a Cₘₐₓ in the subject of between about 0.01p M and about 10 μM. In some cases, the colorectal cancer is colon cancer, sigmoid colon cancer, adenocarcinoma of the colon, stage III colorectal cancer, stage IV colorectal cancer, or descending colon cancer. The dosage of endoxifen may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating colorectal cancer (e.g., colon cancer, sigmoid colon cancer, adenocarcinoma of the colon, stage III colorectal cancer, stage IV colorectal cancer, or descending colon cancer) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating colorectal cancer maintains a concentration of systemic endoxifen above about 0.1 μM for at least one week. In some embodiments, method for treating colorectal cancer maintains a concentration of systemic endoxifen above about 0.5 μM for at least one week. In some embodiments, method for treating colorectal cancer maintains a concentration of systemic endoxifen above about 1 μM for at least one week. In some embodiments, method for treating colorectal cancer maintains a concentration of systemic endoxifen above about 2 μM for at least one week.

In some embodiments, a dosage of endoxifen for treating gastric cancer may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating gastric cancer may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating gastric cancer maintains a concentration of systemic endoxifen above about 0.1 μM for at least one week. In some embodiments, method for treating gastric cancer maintains a concentration of systemic endoxifen above about 0.5 μM for at least one week. In some embodiments, method for treating gastric cancer maintains a concentration of systemic endoxifen above about 1 μM for at least one week. In some embodiments, method for treating gastric cancer maintains a concentration of systemic endoxifen above about 2 μM for at least one week.

In some embodiments, a dosage of endoxifen for treating neuroblastoma may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating neuroblastoma may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating neuroblastoma maintains a concentration of systemic endoxifen above about 0.1 μM for at least one week. In some embodiments, method for treating neuroblastoma maintains a concentration of systemic endoxifen above about 0.5 μM for at least one week. In some embodiments, method for treating neuroblastoma maintains a concentration of systemic endoxifen above about 1 μM for at least one week. In some embodiments, method for treating neuroblastoma maintains a concentration of systemic endoxifen above about 2 μM for at least one week.

In some embodiments, a dosage of endoxifen for treating pancreatic cancer (e.g., pancreatic ductal adenocarcinoma, metastatic pancreatic ductal adenocarcinoma, or stage III metastatic pancreatic ductal adenocarcinoma) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating pancreatic cancer (e.g., pancreatic ductal adenocarcinoma, metastatic pancreatic ductal adenocarcinoma, or stage III metastatic pancreatic ductal adenocarcinoma) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating pancreatic cancer maintains a concentration of systemic endoxifen above about 0.1 μM for at least one week. In some embodiments, method for treating pancreatic cancer maintains a concentration of systemic endoxifen above about 0.5 μM for at least one week. In some embodiments, method for treating pancreatic cancer maintains a concentration of systemic endoxifen above about 1 μM for at least one week. In some embodiments, method for treating pancreatic cancer maintains a concentration of systemic endoxifen above about 2 μM for at least one week.

In some embodiments, a dosage of endoxifen for treating esophageal cancer (e.g., esophageal adenocarcinoma or metastatic esophageal cancer) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating esophageal cancer (e.g., esophageal adenocarcinoma or metastatic esophageal cancer) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating esophageal cancer maintains a concentration of systemic endoxifen above about 0.1 μM for at least one week. In some embodiments, method for treating esophageal cancer maintains a concentration of systemic endoxifen above about 0.5 μM for at least one week. In some embodiments, method for treating esophageal cancer maintains a concentration of systemic endoxifen above about 1 μM for at least one week. In some embodiments, method for treating esophageal cancer maintains a concentration of systemic endoxifen above about 2 μM for at least one week.

In some embodiments, a dosage of endoxifen for treating rectal cancer (e.g., rectum adenocarcinoma) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating rectal cancer (e.g., rectum adenocarcinoma) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating rectal cancer maintains a concentration of systemic endoxifen above about 0.1 μM for at least one week. In some embodiments, method for treating rectal cancer maintains a concentration of systemic endoxifen above about 0.5 μM for at least one week. In some embodiments, method for treating rectal cancer maintains a concentration of systemic endoxifen above about 1 μM for at least one week. In some embodiments, method for treating rectal cancer maintains a concentration of systemic endoxifen above about 2 μM for at least one week.

In some embodiments, a dosage of endoxifen for treating cholangiocarcinoma (e.g., metastatic cholangiocarcinoma) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating cholangiocarcinoma (e.g., metastatic cholangiocarcinoma) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating cholangiocarcinoma maintains a concentration of systemic endoxifen above about 0.1 μM for at least one week. In some embodiments, method for treating cholangiocarcinoma maintains a concentration of systemic endoxifen above about 0.5 μM for at least one week. In some embodiments, method for treating cholangiocarcinoma maintains a concentration of systemic endoxifen above about 1 μM for at least one week. In some embodiments, method for treating cholangiocarcinoma maintains a concentration of systemic endoxifen above about 2 μM for at least one week.

In some embodiments, a dosage of endoxifen for treating breast cancer (e.g., triple negative breast cancer, metastatic breast cancer, or HER2+ breast cancer) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating breast cancer (e.g., triple negative breast cancer, metastatic breast cancer, or HER2+ breast cancer) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating breast cancer maintains a concentration of systemic endoxifen above about 0.1 μM for at least one week. In some embodiments, method for treating breast cancer maintains a concentration of systemic endoxifen above about 0.5 μM for at least one week. In some embodiments, method for treating breast cancer maintains a concentration of systemic endoxifen above about 1 μM for at least one week. In some embodiments, method for treating breast cancer maintains a concentration of systemic endoxifen above about 2 μM for at least one week.

In some embodiments, a dosage of endoxifen for treating cervical cancer (e.g., cervical carcinoma) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating cervical cancer (e.g., cervical carcinoma) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating cervical cancer maintains a concentration of systemic endoxifen above about 0.1 μM for at least one week. In some embodiments, method for treating cervical cancer maintains a concentration of systemic endoxifen above about 0.5 μM for at least one week. In some embodiments, method for treating cervical cancer maintains a concentration of systemic endoxifen above about 1 μM for at least one week. In some embodiments, method for treating cervical cancer maintains a concentration of systemic endoxifen above about 2 μM for at least one week.

In some embodiments, a dosage of endoxifen for treating ovarian cancer (e.g., malignant endometrioid ovarian carcinoma, stage IIIC poorly-differentiated serous adenocarcinoma, fallopian tube carcinoma, stage IV ovarian cancer, granulosa cell tumor of ovary, high grade carcinoma of Mullerian/ovarian origin, platinum-resistant recurrent ovarian cancer, high grade serous carcinoma, ovarian mucinous adenocarcinoma, metastatic ovarian cancer, low grade serous ovarian cancer, recurrent ovarian cancer, or peritoneal carcinomatosis) may be selected to achieve a Cₘₐₓ in the subject of at least about 0.01 μM, at least about 0.05 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, or at least about 10 μM. In some embodiments, a dosage of endoxifen for treating ovarian cancer (e.g., malignant endometrioid ovarian carcinoma, stage IIIC poorly-differentiated serous adenocarcinoma, fallopian tube carcinoma, stage IV ovarian cancer, granulosa cell tumor of ovary, high grade carcinoma of Mullerian/ovarian origin, platinum-resistant recurrent ovarian cancer, high grade serous carcinoma, ovarian mucinous adenocarcinoma, metastatic ovarian cancer, low grade serous ovarian cancer, recurrent ovarian cancer, or peritoneal carcinomatosis) may be selected to achieve a Cₘₐₓ in the subject of from about 0.01 μM to about 50 μM, from about 0.01 μM to about 0.1 μM, from about 0.1 μM to about 0.5 μM, from about 0.1 μM to about 1 μM, from about 0.1 μM to about 2 μM, from about 0.5 μM to about 1 μM, from about 0.5 μM to about 2 μM, from about 1 μM to about 2 μM, from about 1 μM to about 5 μM, or from about 1 μM to about 10 μM. In some embodiments, method for treating ovarian cancer maintains a concentration of systemic endoxifen above about 0.1 μM for at least one week. In some embodiments, method for treating ovarian cancer maintains a concentration of systemic endoxifen above about 0.5 μM for at least one week. In some embodiments, method for treating ovarian cancer maintains a concentration of systemic endoxifen above about 1 μM for at least one week. In some embodiments, method for treating ovarian cancer maintains a concentration of systemic endoxifen above about 2 μM for at least one week.

In some embodiments, a method of treating a cancer (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) may comprise administering (e.g., orally, topically, or via inhalation) an endoxifen composition to the subject's plasma endoxifen at steady state levels greater than 30 nM, for example, at levels ranging from 30 nM to 80 nM or at levels ranging from 30 nM to 300 nM. In some embodiments, the plasma steady state endoxifen levels are maintained at >40 nM. Maintenance of such a plasma endoxifen at steady state levels greater than 30 nM is advantageous in that the likelihood of recurrence (relapse) of hormone-dependent breast disorders or hormone-dependent reproductive tract disorders at plasma endoxifen levels lower than 30 nM is reduced. It is particularly advantageous for subjects that are poor metabolizers of tamoxifen (with plasma endoxifen levels lower than 16 nM), intermediate metabolizers of tamoxifen (with plasma endoxifen levels lower than 27 nM) to be dosed with a composition disclosed herein. It also advantageous for subjects being treated or to be treated with antidepressant drugs such as SSRI drugs such as citalopram (Celexa), escitalopram (Lexapro), fluoxetine (Prozac), paroxetine (Paxil, Pexeva), sertraline (Zoloft), vilazodone (Viibryd) and the like, for example, a subject having or likely to have depression.

In some embodiments, a steady state endoxifen level (e.g., steady state plasma endoxifen level) may be maintained at a level relative to an IC₅₀ of endoxifen to the cancer type of interest (e.g., melanoma, colorectal cancer, gastric cancer, neuroblastoma, pancreatic cancer, esophageal cancer, rectal cancer, cholangiocarcinoma, breast cancer, cervical cancer, or ovarian cancer). For example, a steady state of endoxifen may be maintained at no less than 0.0001, no less than 0.0002, no less than 0.0003, no less than 0.0004, no less than 0.0005, no less than 0.0006, no less than 0.0007, no less than 0.0008, no less than 0.0009, no less than 0.001, no less than 0.002, no less than 0.003, no less than 0.004, no less than 0.005, no less than 0.006, no less than 0.007, no less than 0.008, no less than 0.009, or no less than 0.01 times the IC₅₀ of endoxifen to the cancer type of interest. In some embodiments, the plasma level of endoxifen may be maintained for at least 7, at least 14, at least 21, or at least 28 days, thereby treating a cancer (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) in the subject.

Whether a subject is tamoxifen-refractory may be determined by dosing a subject with an initial dosage of tamoxifen and determining the subject's plasma endoxifen steady state level. Plasma endoxifen steady state levels in a subject dosed with tamoxifen serves as a biomarker for the tamoxifen-refractory subjects. The plasma endoxifen levels (acute and/or steady state) may be determined by obtaining from the subject a test sample, which may be blood sample, collected from the subject after dosing the subject with tamoxifen. Plasma or serum may be obtained from blood samples for testing the biomarker endoxifen levels. The initial dosage may comprise administering tamoxifen daily for at least 1 day, 2 days, 3 days, 15 days, 1 week, 2 weeks, 4 weeks, 1 month, 2 months, 3 months, 4 months, 5 months, or 6 months. The subject may also be administered with a first composition comprising tamoxifen daily for at least 1 day, 2 days, 3 days, 15 days, 1 week, 2 weeks, 4 weeks, 1 month, 2 months, 3 months, 4 months, 5 months, 6 months, 1 year, 2 years, 3 years, 4 years, 5 years or 10 years.

A subject's plasma endoxifen steady state level may be determined by measuring endoxifen in a test sample. The subject's plasma endoxifen steady state levels are compared to a reference plasma endoxifen level. For the purposes of the present disclosure, the reference plasma level is 30 nM. If the subject's plasma endoxifen level is determined to be lower than 30 nM, then the subject is defined as tamoxifen-refractory. Such a tamoxifen-refractory subject who has or who may be at risk of having a hormone-dependent breast disorder or hormone-dependent reproductive tract disorder is treated by administering to the subject an oral composition comprising (Z)-endoxifen or a salt thereof disclosed herein, or a polymorphic form of endoxifen disclosed herein. In some embodiments, the composition administered to such a subject comprises (Z)-endoxifen free base. In other embodiments, the composition administered to such a subject comprises endoxifen gluconate selected from the group consisting of (Z)-endoxifen D-gluconate, (Z)-endoxifen L-gluconate, (E)-endoxifen D-gluconate, (E)-endoxifen L-gluconate, or a combination thereof. In other embodiments, the composition comprising endoxifen is endoxifen HCl or endoxifen citrate. The present disclosure also contemplates that a subject's plasma endoxifen levels are tracked or monitored periodically or as necessary. If required, a subject who has been administered an initial dosage of tamoxifen may have his or her plasma endoxifen steady state levels adjusted by administering a composition comprising endoxifen on an ongoing basis based on the test results.

In some embodiments, the subject's tamoxifen-refractory status may be determined by determining the subject's tamoxifen-metabolites profile which is compared with a reference tamoxifen-metabolite profile as seen in control or normal subjects. Subjects with low plasma endoxifen levels in subject's tamoxifen-metabolite profile as compared to the reference tamoxifen-metabolite profile are administered an oral composition comprising endoxifen or a salt thereof. Such compositions may comprise synthetically prepared endoxifen.

The plasma endoxifen may be measured by any of method known in the art. The levels of plasma endoxifen in test sample may be determined based on subject's genes, DNA, RNA, protein, tamoxifen-metabolite profile, or a combination thereof. The tamoxifen-metabolites profile can include at least tamoxifen, 4-OHT, N-desmethyltamoxifen, and/or endoxifen. In some embodiments, the level of plasma endoxifen and/or tamoxifen-metabolite profile in the test sample is measured by High Performance Liquid Chromatography (HPLC), Gas Chromatography Mass Spectrometry (GC-MS), Liquid Chromatography Mass spectrometry (LC-MS), Liquid Chromatography Tandem Mass spectrometry (LC-MS/MS), immunohistochemistry (IHC), polymerase chain reaction (PCR), quantitative PCR (qPCR), and the like. In some embodiments, the tamoxifen-metabolites profile is predicted based on the subject's genetic composition. In some embodiments, the subject's CYP genotype includes, without limitation, analysis of CYP2D6, CYP3A4, CYP2C9 genes. In some embodiments, subject's estrogen receptor levels may be analyzed. In other embodiments, the determination of plasma endoxifen may be done by a third-party laboratory.

Accordingly, provided herein are methods of maintaining in a subject in need thereof a plasma endoxifen a level greater than 30 nM by administering to the subject a composition comprising endoxifen or a salt thereof. In some embodiments, the subject's plasma endoxifen level is maintained at a steady state level greater than 30 nM. In some embodiments, the subject's plasma endoxifen levels are maintained at a steady state level ranging from 30 nM to 300 nM (for example, from 30 nM to 200 nM, or from 30 nm to 80 nM). In some embodiments, the subject's plasma endoxifen levels are maintained at a steady state level>40 nM. In some embodiments, the plasma level of endoxifen may be maintained for at least 7, at least 14, at least 21, or at least 28 days, thereby treating a cancer (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) in the subject.

In another aspect, the subjects may have their test samples tested for their biomarker profile that may be indicative or monitoring a hormone-dependent breast disorder, a hormone-dependent reproductive tract disorder, or both. Such biomarkers are known in the art and include, by way of non-limiting examples, biomarkers such as CYP2D6, BRCA-1, BRCA-2, ER, PR, Her2, uPA, PAI, Tf, p53, Ki67, cytokeratins, cancer tumor antigens, and other biomarkers measured by Mammaprint, OncotypeDx, PAM50, EndoxPredict, MammoStrat, and other diagnostic and predictive tests. A subject with biomarker profile indicating that the subject has or is at risk of having a hormone-dependent breast disorder, a hormone-dependent reproductive tract disorder, or both can be administered a composition disclosed herein. In one aspect, the present disclosure provides a method of treating a subject having or at risk of having a hormone-dependent breast disorder, a hormone-dependent reproductive tract disorder, or both, comprising determining a subject's tamoxifen-refractory or tamoxifen-resistant status and administering to the subject a composition described herein.

### (v) Metastatic Cancer

Aspects of the present disclosure provide methods of treating a metastatic cancer comprising administering a therapeutically effective amount of endoxifen to a subject in need thereof. A surprising discovery disclosed herein is that endoxifen can be effective for treating certain metastatic cancers, including some metastatic skin cancers, metastatic gastrointestinal cancers, and metastatic neuroblastomas. Metastatic cancers often exhibit oncogenetic and phenotypic diversity, broad tissue distributions, and unresponsiveness to primary treatments. These characteristics can limit the efficacies of targeted and immunomodulatory treatments, and can thereby necessitate the use of potent anti-cancer agents. The low IC₅₀ values identified for endoxifen against many of the cancers disclosed herein, including some metastatic cancers (e.g., metastatic pancreatic ductal adenocarcinoma as outlined in EXAMPLE 12), suggest that endoxifen can be an effective treatment option for some metastatic cancers.

### (vi) Operative and Post-Operative Endoxifen Courses

In another example, an endoxifen composition may be administered in combination with a surgical or radiological treatment. For many of the methods disclosed herein, endoxifen is administered prior to, concurrently with, or subsequent to surgical tumor or organ removal (e.g., cholecystectomy) or radiation therapy. Cancer recurrence following such treatments often stems from small remaining metastases, incomplete removal of the cancerous tissue, or remaining portions of cancerous or precancerous tissue. Owing to its low IC₅₀ for many cancers, endoxifen can provide an effective means for clearing remaining cancer cells following surgical or radiological treatment.

Endoxifen can be an effective post-operative treatment for clearing remaining cancer cells for cancers in which other therapies are unable to clear remaining cancer fractions. In many cases, therapies which rely on immune-mediated cancer cell clearance, for example some immune-activating therapies for PD-L1 and CD155 positive cancers and certain chemotherapies, have low efficacies against precancerous cells and small metastases with low immune cell densities. As identified herein, endoxifen can directly affect cancer cell death and clearance without concomitant activation or participation from immune cells. For these cancers, endoxifen administration can be a preferred method for clearing remaining cancer cells or suppressing further metastasis following surgery and/or radiation therapy.

A method disclosed herein can comprise administering to a subject a therapeutically effective amount of endoxifen and an additional therapy. In some cases, the additional therapy is surgical tumor removal, radiation therapy, or a combination thereof. In some cases, the administering the therapeutically effective amount of endoxifen is prior to the surgical tumor removal and/or radiation therapy, concurrent with the surgical tumor removal and/or radiation therapy, subsequent to the surgical tumor removal and/or radiation therapy, or a combination thereof. In some cases, the administering the therapeutically effective amount of endoxifen is subsequent to the surgical tumor removal and/or radiation therapy.

Surgical tumor removal can comprise complete (e.g., “total resection”) or partial (e.g., “debulking”) of a tumor and/or cancerous tissue in a subject. In some cases, surgical tumor removal comprises cryosurgery, which hereinafter can refer to methods which kill or weaken cancerous tissue with cooling. In some cases, surgical tumor removal comprises laser removal, in which high energy coherent light is used to damage, kill, or excise cancerous tissue. In some cases, surgical tumor removal is laparoscopic.

A radiation therapy as disclosed herein can include, without limitation, external-beam radiation therapy, brachytherapy (e.g., implant-based radiation therapy), systemic radiation therapy, stereotactic radiosurgery, and combinations thereof. In some cases, the radiation therapy utilizes x-rays or gamma rays. In some cases, the radiation therapy utilizes particles such as electrons, protons, neutrons, carbon ions, alpha particles, and/or beta particles. In some cases, the radiation therapy provides a cumulative external radiation dose of about 1 to about 100 Grays (“Gy”, the equivalent of 10² roentgen equivalent man (rem)). As used herein, a cumulative external radiation dose can refer to the total amount of radiation provided to a subject or to a treated area over a full course of treatment. In some cases, the radiation therapy provides a cumulative external radiation dose of about 1 to about 20 Gy, of about 1 to about 40 Gy, of about 5 to about 30 Gy, of about 10 to about 40 Gy, of about 10 to about 50 Gy, of about 20 to about 60 Gy, or about 5 to about 25 Gy. In some cases, the radiation therapy is administered in one dose. In some cases, the radiation therapy is administered in 1 to 5 doses, in 1 to 10 doses, in 3 to 10 doses, in 4 to 15 doses, in 6 to 20 doses, in 4 to 12 doses, or in 6 to 15 doses (e.g., in equivalent doses). The doses can be administered hourly, daily, once every 1-10 days, once every 1-14 days, once every 3-14 days, once every 7-31 days, or once every 14-31 days.

### Endoxifen Formulations

A method of treating a cancer may comprise administering a composition comprising endoxifen to the subject. Endoxifen, also referred to as 4-hydroxy-N-desmethyl-tamoxifen, may include a polymorphic, salt, free base, co-crystal, or solvate form of endoxifen. In some embodiments, the endoxifen may comprise one or more polymorphic forms, such as Form I, Form II, or Form III, of endoxifen. A polymorphic form may be distinguished by its x-ray powder diffraction pattern. In some embodiments, a method of treating a cancer may comprise administering a pharmaceutical composition comprising endoxifen predominantly as polymorph Form I. In some embodiments, the pharmaceutical composition comprises endoxifen predominantly as polymorph Form II. In some embodiments, the pharmaceutical composition comprises endoxifen predominantly as polymorph Form III.

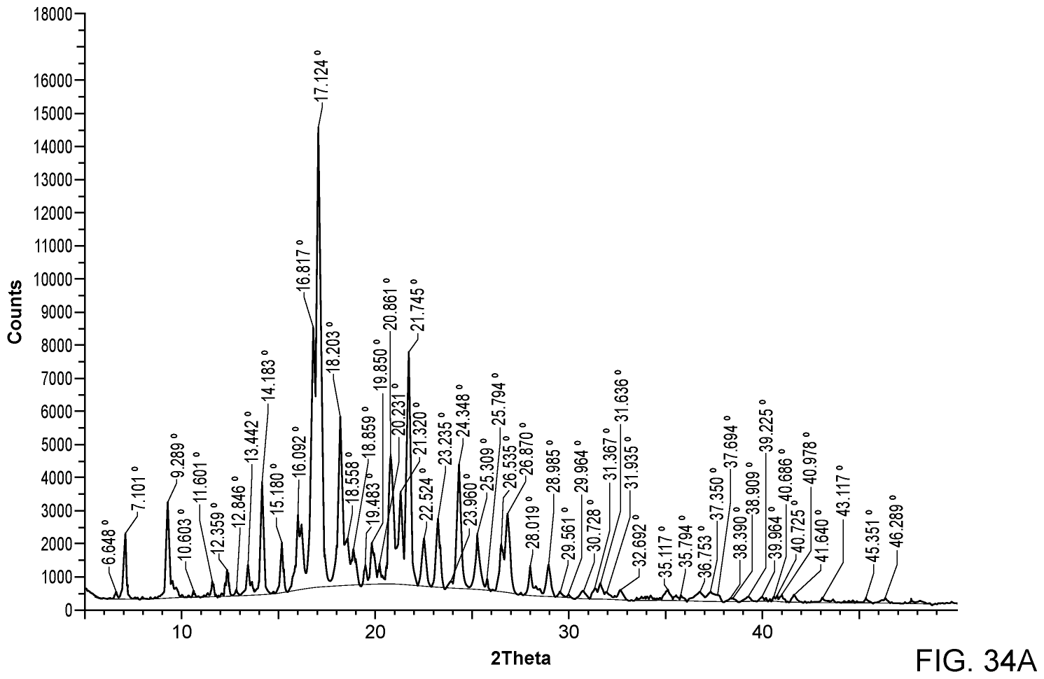

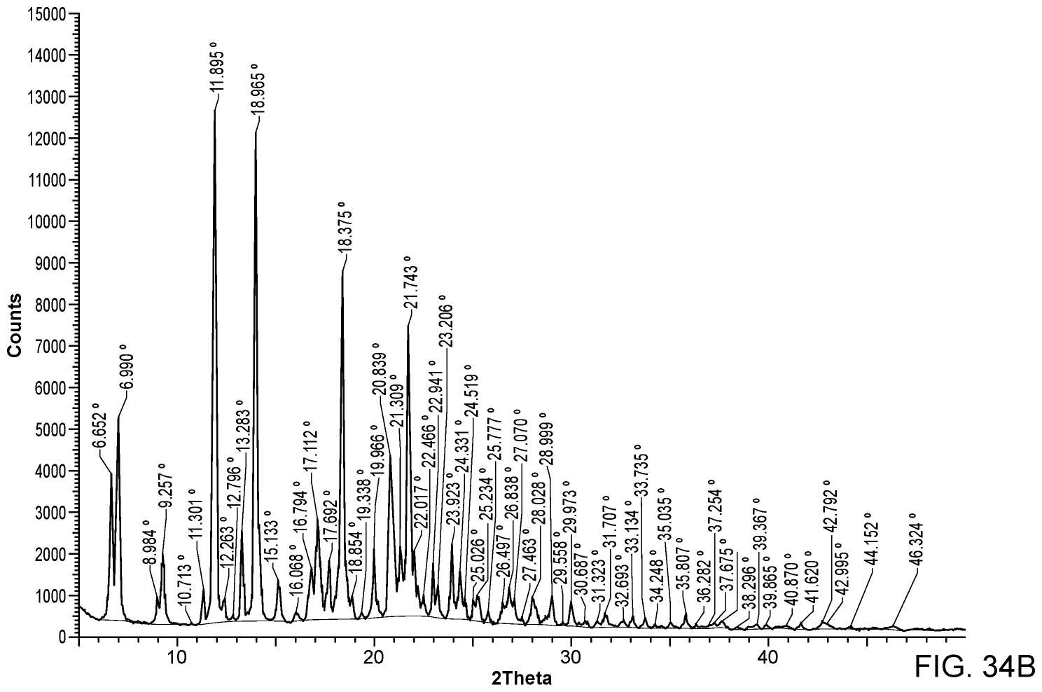

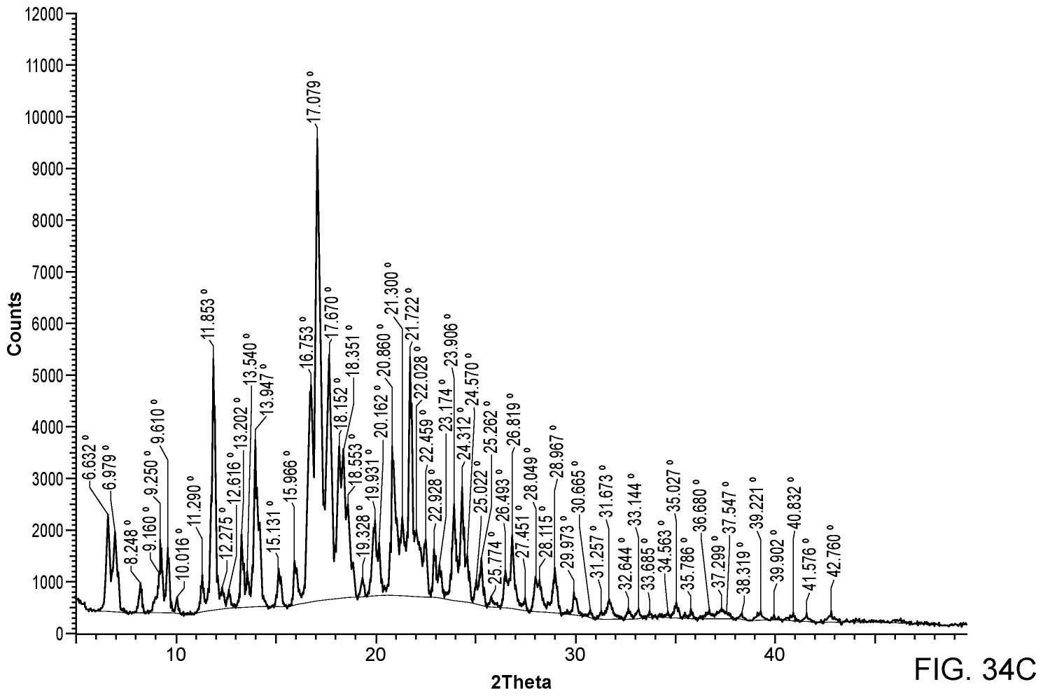

In some embodiments, polymorphic Form I is characterized by an x-ray powder diffraction pattern comprising major peaks at 16.8±0.3°, 17.1±0.3° and 21.8±0.3° two theta. In some embodiments, crystalline Form I is characterized by an x-ray powder diffraction pattern substantially as set forth in FIG. 34A. In some embodiments, polymorphic Form II is characterized by an x-ray powder diffraction pattern comprising major peaks at 7.0±0.3°, 11.9±0.3°, 14.0±0.3° and 18.4±0.3° two theta. In some embodiments, crystalline Form II is characterized by an x-ray powder diffraction pattern substantially as set forth in FIG. 34B. In some embodiments, polymorphic Form III is characterized by an x-ray powder diffraction pattern comprising major peaks at 11.9±0.3°, 13.9±0.3°, 17.1±0.3° and 17.7±0.3° two theta. In some embodiments, crystalline Form III is characterized by an x-ray powder diffraction pattern substantially as set forth in FIG. 34C.

In some embodiments, a composition comprises endoxifen as at least 0.1%, at least 0.2%, at least 0.3%, at least 0.4%, at least 0.5%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, or 100% of a single polymorphic Form of endoxifen, such as Form I, Form II, or Form III, wt/wt of total endoxifen in the composition. In some embodiments, a composition for treating various cancers comprises ≥90% of a single polymorphic Form of endoxifen, such as Form I, Form II, or Form III, wt/wt of the total endoxifen in the composition. In another embodiment, the composition comprises ≥95% of a single polymorphic Form of endoxifen, such as Form I, Form II, or Form III, wt/wt of the total endoxifen in the composition. In some embodiments, the composition comprises ≥96%, ≥97%, ≥98%, ≥99%, or ≥99.5% of a single polymorphic Form of endoxifen, such as Form I, Form II, or Form III, wt/wt of the total endoxifen in the composition. When a particular percentage by weight of endoxifen is a single polymorphic form, the remainder of endoxifen in the composition may be some combination of amorphous endoxifen and/or one or more polymorphic forms of endoxifen excluding the single polymorphic form. When the polymorphic endoxifen is defined as one particular form of endoxifen, the remainder may be made up of amorphous endoxifen and/or one or more polymorphic forms other than the particular form specified. Examples of single polymorphic forms include Forms I, II and III of endoxifen, as well as descriptions of a single polymorphic form characterized by one or more properties as described herein.

In other embodiments, a composition comprising endoxifen comprises 0.01% to 20%, 0.05% to 15%, or 0.1% to 10% of a single polymorphic Form of endoxifen, such as Form I, Form II, or Form III, wt/wt or w/v of the composition. In at least one embodiment, the composition comprising endoxifen comprises 0.01% to 20% of a single polymorphic Form of endoxifen, such as Form I, Form II, or Form III, wt/wt or w/v of the composition. In various other embodiments, the composition comprising endoxifen comprises 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% of a single polymorphic Form of endoxifen, such as Form I, Form II, or Form III, wt/wt of the composition. In an aspect, a composition comprising a single polymorphic Form of endoxifen, such as Form I, Form II, or Form III, further comprises a second polymorphic Form of endoxifen.

In some embodiments, an endoxifen composition may comprise no more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% (wt/wt) of E-endoxifen, relative to total endoxifen. In some embodiments, an endoxifen composition may comprise no less than 99.9%, 99%, 98%, 97%, 96%, 95%, 90%, 85%, 80%, or 70% (wt/wt) of Z-endoxifen, relative to total endoxifen. In some embodiments, an endoxifen composition may comprise no more than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% (wt/wt) of impurities, relative to Z-endoxifen. In some embodiments, an endoxifen composition may comprise no less than 99.9%, 99%, 98%, 97%, 96%, 95%, 90%, 85%, 80%, or 70% (wt/wt) of Z-endoxifen, relative to total composition. In some embodiments, an endoxifen composition comprises no less than 99.9%, 99%, 98%, 97%, 96%, 95%, 90%, 85%, 80%, or 70% (wt/wt) of Z-endoxifen, relative to total endoxifen (e.g., comprises an isomeric purity of at least 70%, at least 80%, at least 85%, at least 90%, at least 95%, at least 96%, at least 97%, at least 98%, at least 99%, or at least 99.9% for (Z)-endoxifen).

### Pharmaceutical Compositions

A composition for treatment of various cancers may be formulated for as a pharmaceutical composition. For example, a composition comprising endoxifen (e.g., Z-endoxifen) may be formulated as a pharmaceutical composition. In some embodiments, a composition for treating various cancers (e.g., melanoma, colorectal cancer, gastric cancer, neuroblastoma, pancreatic cancer, esophageal cancer, rectal cancer, cholangiocarcinoma, breast cancer, cervical cancer, or ovarian cancer) may comprise endoxifen (e.g., Z-endoxifen). In some embodiments, a pharmaceutical composition may further comprise an additional therapeutic agent. For example, a pharmaceutical composition may comprise an anti-cancer agent. In some embodiments, the anti-cancer agent may be bicalutamide, enzalutamide, trastuzumab, atezolizumab, alpelisib, olaparib, talazoparib, ribociclib, neratinib, an antineoplastic, capecitabine, carboplatin, cisplatin, cyclophosphamide, docetaxel, doxorubicin, pegylated liposomal doxorubicin, epirubicin, fluorouracil, gemcitabine, methotrexate, paclitaxel, protein-bound paclitaxel, vinorelbine, eribulin, ixabepilone, an immune checkpoint inhibitor, a PD1 inhibitor, a PD-L1 inhibitor, a CTLA4 inhibitor, or an ATP-cassette binding protein inhibitor. In some embodiments, the pharmaceutical composition may comprise a selective serotonin reuptake inhibitor (e.g., citalopram, escitalopram, fluoxetine, paroxetine, sertraline, or vilazodone).

In some embodiments, a pharmaceutical composition (e.g., a pharmaceutical composition comprising endoxifen) may be formulated for oral, topical, rectal, intravenous, intra-arterial, parenteral, or transdermal administration, or for administration via inhalation. In some embodiments, an endoxifen composition may be formulated as a sustained-release composition or a delayed release composition. In some embodiments, an endoxifen composition may be formulated as a capsule or a tablet. In some embodiments, an endoxifen composition may be formulated as a cream, a gel, a cream, an emulsion, a lotion, an ointment, a solution, a paste, a patch, or an oil.

In various embodiments, a pharmaceutical composition provided herein comprises from about 1% to about 99.99%, about 5% to about 95%, about 5% to about 90%, about 10% to about 80%, about 15% to about 70%, about 20% to about 60%, from about 30% to about 95%, from about 50% to about 90%, from about 60% to about 90%, from about 60% to about 80%, or from about 70% to about 80% by weight of one or more excipients. In certain embodiments, the composition provided herein comprises about 99.99%, about 95%, about 90%, about 85%, about 80%, about 75%, about 70%, about 65%, about 60%, about 55%, or about 50% by weight of one or more excipients. In certain embodiments, the composition provided herein comprises about 99.99%, about 99%, about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions formulated for oral administration are provided herein and can include, but are not limited to, one or more of bulking agents, binders, fillers, disintegrating agents, lubricants, glidants, control release agents, enteric coatings, film-forming agents, plasticizers, colorants, sweeteners, flavoring agents and the like, or any combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, methocel, walocel HM, Luvitec, Luvicaparolactam, Avicel, SMCC, UNIPURE, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

One or more binder or filler in compositions is typically present in from about 10% to about 99% (wt/wt) of the composition or the dosage form. In some embodiments, binders and/or fillers in a composition comprise about 15% to 99%, about 20% to 60%, about 25% to 55%, about 30% to 50%, about 35% to 60%, about 50% to 99% (wt/wt) of the composition.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant is deep in the oral solid dosage form to delay disintegration. The amount of disintegrant used varies based upon the type of formulation, and is readily discernible to those of ordinary skill in the art.

In some embodiments, compositions comprise from 0.5% to 15% (wt/wt) of disintegrant. In some embodiments, compositions comprise from 1% to 5% (wt/wt) of disintegrant in the composition. In another embodiment, the disintegrant is 1% to 25%, 2% to 20%, 5% to 15%, 8% to 12%, or about 10% (wt/wt) of the composition.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), Q7-9120 (Dow Corning), and mixtures thereof. If used at all, lubricants are typically used in an amount of less than 1% (wt/wt) of the compositions or dosage forms into which they are incorporated. In yet another embodiment, the lubricant is 0.1% to 3%, such as 0.5% to 1% (wt/wt), of the composition.

Plasticizers may be added to control the softness or pliability of oral dosage forms such as shell of a capsule, caplet, or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

As an example of sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearmint, methyl salicylate, raspberry, red berry, strawberry, pineapple, orange, cherry, and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, a pharmaceutical composition comprising endoxifen (e.g., (Z)-endoxifen) may be formulated as a sustained release composition. In some embodiments, a pharmaceutical composition comprising a phosphoinositide 3-kinase inhibitor (e.g., alpelisib) may be formulated as a sustained release composition. Sustained release agent present in a sustained release composition of the present disclosure may be any sustained release agent known in the art to slow the release of a hydrophobic drug such as (Z)-endoxifen or a polymorph or a salt thereof.

Examples of sustained release agents include cellulosic ethers, gums, acrylic resins such as polymers and copolymers of acrylic acid, methacrylic acid, methyl acrylate, methyl methylacrylate, and combinations thereof, polyvinyl pyrrolidine, and protein-derived compounds. Examples of cellulosic ethers include hydroxyalkyl celluloses, hydroxyethyl celluloses, hydroxypropyl celluloses, hydroxypropylmethyl celluloses (HPMC or hypromellose, for example Nos. 2208, 2906, 2910), carboxyalkyl celluloses, and carboxymethyl celluloses. In some embodiments, the at least one sustained release agent is a pH sustained release agent such as acid insoluble polymers which become increasingly soluble and permeable above pH 5.0 but remaining impermeable below pH 5.0. Such controlled release polymers target upper small intestines and/or colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers, including those available commercially from Evonik or Rohm ((Eudragit® sustained release polymers Eudragit® RL (high permeability), Eudragit® RS (low permeability) and Eudragit® NM 30D (low permeability)—alone or in any combination thereof to achieve the desired permeability for sustained release. The viscosity of sustained release agents may be any viscosity suitable for sustained release of (Z)-endoxifen or a polymorph or a salt thereof. In certain embodiments, the viscosity of the at least sustained release agent ranges from about 1000 mPa·s to about 150,000 mPa·s. In some embodiments, the sustained release delivery system includes one or more SR/release rate controlling agents with viscosity ranging from about 1000 mPa·s to about 10,000 mPa·s, from about 10,000 mPa·s to about 70,000 mPa·s, from about 70,000 mPa·s to about 150,000 mPa·s. or a combination thereof. In some embodiments, the present disclosure provides that the sustained release delivery system includes two or more sustained release agents. Each sustained release agent may have the same viscosity or a differing viscosity, for example one sustained release agent may have a viscosity ranging from about 1000 mPa·s to about 10,000 mPa·s, while other sustained release agent may have a viscosity of about 10,000 mPa·s to about 70,000 mPa·s or about 70,000 mPa·s to about 150,000 mPa·s.

In some embodiments, the sustained release agent is HPMC/hypromellose (e.g., Nos. 2208, 2906, 2910). Hypromellose to be used in the present disclosure has a weight molecular average of about 20,000-500,000. In some embodiments, hypromellose has a molecular weight average of generally 20,000-250,000. Hypromellose is commercially available from Dow Chemicals under the trade name Methocell™, for example, Methocell™ K100 (average molecular weight 26,000, 2% viscosity; 75,000-140,000 mPa·s); Methocell™ K15M (average molecular weight 120,000, 2% viscosity; 15,000 cP, 13275-24,780 mPa·s); Methocell™ K4M (average molecular weight 86,000, 2% viscosity; 4,000 cP, 75,000-140,000 mPa·s). Hypromellose of one grade may be used alone or in combination with another grade.

When a sustained release composition showing release of (Z)-endoxifen or a polymorph or a salt thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16, at least 18 hours, at least 24 hours, at least 48 hours, and at least 72 hours is obtained, in some embodiments the sustained release agent, such as Hypromellose, has an average molecular weight generally ranging from 15,000 to 140,000 Daltons. In at least one embodiment, the average molecular weight of about 15,000 Daltons.

The amount of sustained release agent in the composition may be any amount effective to delay the release of the therapeutic agent (Z)-endoxifen, or a polymorph or a salt thereof, for about 2 hours post-dose to protect the therapeutic agent from the acidic environment of the stomach and allow passage of the therapeutic agent through the stomach into the intestines and prolong such release for a period of about 2 hours to about 72 hours. The amount of sustained release agent in the composition may be any amount effective to provide a slower rate of release of the therapeutic agent (Z)-endoxifen, or a polymorph or a salt thereof as compared with the reference product. In some embodiments, the amount of sustained release agent in the composition may be any amount effective to delay the release of the therapeutic agent (Z)-endoxifen, or a polymorph or a salt thereof, for at least about 1 hour, at least about 1.1 hours, at least about 1.2 hours, at least about 1.3 hours, at least about 1.4 hours, at least about 1.5 hours, at least about 1.6 hours, at least about 1.7 hours, at least about 1.8 hours, at least about 1.9 hours, at least about 2 hours, at least about 2.1 hours, at least about 2.2 hours, at least about 2.3 hours, at least about 2.4 hours, or at least about 2.5 hours post-dose, as compared with the reference product.

When a sustained release composition shows percentage dissolution ranging from about 0% to 35% at 3 hours, from about 35% to about 55% at 12 hours, and from about 65% to 85% at 24 hours in a dissolution test according to the 75 RPM USP paddle method and using pH 1.2 at 37° C. for 2 hours in simulated gastric fluid and pH 6.8 at 37° C. for 24 hours in simulated intestinal fluid as a test medium, at least one sustained release agent, such as Hypromellose (HPMC), may generally be present in a sustained release composition of the present disclosure in amounts from about 0.1% to about 99%, from about 0.1% to about 90%, from about 5% to about 90%, from about 5% to about 80%, from about 5% to about 70%, and from about 5% to about 60% w/w of the sustained release composition. In some embodiments, the sustained release agent (e.g., a gum, an acrylic resin, methacrylic acid, methyl acrylate, methyl methylacrylate, polyvinyl pyrrolidine, a protein-derived compound, a hydroxyalkyl cellulose, a hydroxyethyl cellulose, a hydroxypropyl cellulose, a hydroxypropylmethyl celluloses, a carboxyalkyl cellulose, or, a carboxymethyl cellulose) may be present in an amount of from about 10% to about 40%, from about 10% to about 50%, from about 10% to about 60%, from about 20% to about 40%, from about 20% to about 50%, from about 20% to about 60%. In some embodiments, the sustained release agent may be present in an amount of at least about 10%, at least about 20%, at least about 30%, or at least about 40%. Such sustained release compositions of the present disclosure as disclosed herein release (Z)-endoxifen or polymorphs or salts thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, and at least 72 hours.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

For time delay or delayed-release pharmaceutical preparations of oral dosage forms, glyceryl monostearate, glyceryl distearate, and acid-insoluble polymers, for example polymethacrylate pH-sensitive polymer-based coatings can be used, (e.g., as coating material, such as enteric coating agents, for enteric coating of capsules, caplets, and tablets). Commercial sources for delayed-release oral dosage forms are available, for example DRCaps made of hypromellose (HPMC) from Capsugel, USA. Such delayed-release oral dosage forms are acid-resistant and can resist acidity as seen in stomach for at least 30 min, such as for at least 1 hour, for at least 1.5 hour, or for at least 2 hours. Such delayed release oral dosage forms can release at least 40%, at least 50%, at least 60%, at least 70%, at least 80% or at least 90% of the endoxifen or salts thereof in the intestines (small intestines, large intestine/colon, etc.).

In an aspect of the present disclosure, the enteric tablets, enteric caplets, and enteric capsules may be uncoated. Hard uncoated capsules with enteric capability using intrinsically enteric capsule technology (for example, EnTrinsic Drug Delivery available from Capsugel) are suitable for the purpose of the present disclosure.

In various embodiments, the enteric tablet is a hard tablet made with free-flowing powder of (Z)-endoxifen or a salt thereof. In various embodiments, the enteric capsule is a capsule made with free-flowing powder of (Z)-endoxifen or a salt thereof. In various embodiments, the enteric tablet is a hard tablet made with free-flowing powder of endoxifen or a polymorph thereof. In various embodiments, the enteric capsule is a capsule made with free-flowing powder of endoxifen or a polymorph thereof.

In some embodiments, the enteric capsule is a non-animal based capsule, such as a hypromellose capsule (for example, commercially available self-gelling Vcaps, VCaps Plus, VCaps enteric, other enteric capsules made using Xcellodose, ENCODE colonic delivery technology, and EnTrinsic™ drug delivery technology from Capsugel). Other technologies known in the art and available commercially (for example, Qualicaps, USA, Nutrascience, USA, etc.) for the formulating enteric forms of oral solid dosage forms can also be utilized. In at least one embodiment, the capsule is an API-in-capsule, meaning that the (Z)-endoxifen free base or salts thereof is filled neat into the capsule. In such API-in-capsule oral dosage forms, the active ingredient, (Z)-endoxifen or salts thereof can be free flowing powders or micronized powders. When the dosage form is a capsule, in at least one embodiment, the capsule can be a seamless capsule or a banded capsule.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

An endoxifen composition of the present disclosure may be formulated as a pharmaceutical composition for topical or transdermal delivery. In some embodiments, an endoxifen composition for topical or transdermal delivery may be formulated as a cream, a gel, a cream, an emulsion, a lotion, an ointment, a solution, a paste, a patch, or an oil. An endoxifen composition for topical or transdermal delivery may be applied to the skin of a subject to treat a cancer (e.g., melanoma, esophageal cancer, or breast cancer). The topical or transdermal composition may be applied to a region of skin at or near a location of a caner. For example, a topical endoxifen composition may be applied at the site of a cancerous skin growth in a subject with melanoma, thereby treating the melanoma. In another example, a topical endoxifen composition may be applied to the skin around the throat of a subject with esophageal cancer, thereby treating the esophageal cancer. In some embodiments, a pharmaceutical composition for topical or transdermal administration may comprise (Z)-endoxifen, 2-(2-Ethoxyethoxy)ethanol (e.g., Transcutol), isopropanol, a fully saturated emollient triester (e.g., Crodamol GTCC), and mineral oil.

One of skill in the art will further recognize that compositions disclosed herein may comprise one or more of the excipients known in the art and disclosed herein in any combination appropriate for a desired formulation or preparation. Additional excipients may generally be found in Remington's The Science and Practice of Pharmacy, Meade Publishing Co., United States Pharmacopeia/National Formulary. One of skill in the art will be able to select suitable excipients necessary for the preparation of the formulations and appropriate dosage forms compatible with the route of administration based on his or her skill and knowledge in the art and the disclosures made herein. In all cases, the ultimate dosage form should be sterile and stable under the conditions of manufacture and storage.

For formulations of the solid dosage compositions disclosed herein, as the water activity (Aw) is less than 0.75, testing Total Aerobic Plate Count (TAC) and USP indicator organism is typically not necessary. The publication, “Microbial Bioburden on Oral Solid Dosage Form,” by Jose E. Martinez, Pharmaceutical Technology, February 2002, pages 58 to 70, is hereby incorporated by reference in its entirety.

For other formulations, such as liquid or fluid formulations with water activity of less than 0.75, Tests for Specified Microorganisms (S. aureus, Ps. aeruginosa, Salmonella, C. albicans, Clostridia, E. coli and Bile Tolerant Gram negative bacteria) in compliance with USP Guidelines Chapter 62 may not need to be performed.

### Methods of Cancer Detection

A method for treating cancer can include detection, diagnosis, and staging of cancer or precancerous tissue in a subject. Cancer detection can identify the type, distribution, stage, and severity of the cancer, and can be used to determine an optimal treatment course for a subject in need thereof. The cancer detection can also be used for actively monitoring cancer during and subsequent to treatment. In some cases, the cancer detection is used to monitor a subject during remission.

In some cases, a method of cancer detection (e.g., a method of cancer detection as part of a method of treatment) includes tumor imaging. In some cases, the tumor imaging includes computerized tomography, magnetic resonance imaging, positron emission tomography, ultrasound, X-ray imaging, or a combination thereof.

In some cases, a method of cancer detection includes genetic testing. Known cancerous or potentially cancerous tissues can be screened for genetic predispositions (e.g., inherited genetic variations) or mutations ascribable to cancer or elevated risks of developing cancer. In some cases, a method of monitoring or detecting colorectal cancer comprises detecting a mutation in one or more of APC, DCC, TP53, serine/threonine-protein kinase B-Raf (BRAF), phosphatidylinositol-4,5-bisphosphate 3-kinase catalytic subunit alpha (PIK3CA), tumor protein P53 (P53), Kisspeptin proteins (KISS1), Rho family-alpha serine/threonine-protein kinase (AKT1), SMAD4, KRAS, HRAS, and Catenin beta-1 (CTNNB1). In some cases, a method of monitoring or detecting melanoma comprises detecting a mutation in KRAS, HRAS, NRAS, BRAF, CDK4, or a combination thereof. In some cases, a method of monitoring or detecting neuroblastoma comprises detecting a mutation in anaplastic lymphoma kinase (ALK), PHOX2B, FLJ22536, BARD1, NBPF23, or a combination thereof.

In some cases, a method of cancer detection includes expression profiling. In some cases, a method of monitoring or detecting colorectal cancer includes identifying overexpression of one or more of HER2, FOXQ1, CDCP1, NEDD4, Human leukocyte antigen-E protein (HLA-E), HOXC10, HsAIRK1, HsAIRK3, PLAGL2, and CK2a. In some cases, a method of monitoring or detecting melanoma includes identifying overexpression of KRAS, HRAS, NRAS, Cyclin D1, HDM2, NF1, or a combination thereof. In some cases, a method of monitoring or detecting neuroblastoma includes identifying overexpression of ALK, MYCN, or TFAP4, diminished expression of NF1, or a combination thereof.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the terms “a,” “an,” and “the” include plural reference unless the context dictates otherwise.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values ±10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

As used herein, the terms “active pharmaceutical ingredient”, “active ingredient”, “API,” “drug,” “active,” “actives” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present disclosure is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, and/or prevention of disease in man or other animals.

As used herein, “adjuvant therapy” refers to a therapy that follows a primary therapy and that is administered to subjects at risk of relapsing. Adjuvant systemic therapy in case of breast cancer or reproductive tract cancer, for example with tamoxifen, usually begins soon after primary therapy to delay recurrence, prolong survival or cure a subject.

Embodiments that reference throughout this specification to “a compound”, such as compounds of Formula (I), Formula (II), Formula (III) and Formula (IV), include the polymorphic, salt, free base, co-crystal, and solvate forms of the formulas and/or compounds disclosed herein. Thus, the appearances of the phrases “a compound”, “compound of Formula (I)”, “compound of Formula (II)”, “compounds of Formula (III)” and “compound of Formula (IV)” include Form I of the compound of Formula (IV), Forms II-III of the compounds of Formula (III), the free base of the compound of Formula (IV), the free base of the compounds of Formula (III), and/or the gluconate salts as described herein.

The terms “crystalline form”, “polymorph” and “Form” may be used interchangeably herein, and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compound of the present disclosure include crystalline and amorphous forms of those compounds, including, for example, polymorphs, pseudopolymorphs, salts, solvates, hydrates, unsolvated polymorphs (including anhydrates), conformational polymorphs, and amorphous forms of the compounds, as well as mixtures thereof.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹¹C, ¹³C and ¹⁴C.

As used herein and in the claims, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of.”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “pharmaceutically acceptable carrier” or “carrier” means a pharmaceutically acceptable material, composition, or vehicle, such as a liquid or solid filler, diluent, excipient, solvent, or encapsulating material, involved in carrying or transporting one or more of the compounds of the present disclosure from one tissue, organ, or portion of the body or across the skin.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

As used herein, the term “pharmaceutical composition” means a combination of the active agent (e.g., an active pharmaceutical compound or ingredient, API) with a carrier, inert or active (e.g., a phospholipid), making the compositions particularly suitable for diagnostic or therapeutic uses in vitro, in vivo, or ex vivo.

As used herein, the terms “subject,” “patient,” “participant,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

As used herein, the term “cancer” can refer to the presence of cells which exhibit abnormal growth in an organism. The cells often present recognizable expression profiles and morphologies, as well as behaviors such as uncontrolled growth and proliferation, metastatic potential, and immortality. Cancer cells can exist within tumors, within non-cancerous tissue, or apart from other cells (e.g., in circulation or within lymph space).

### EXAMPLES

The invention is further illustrated by the following non-limiting examples.

### Example 1

### Screening Endoxifen in Patient-Derived Tumor Organoids

This example describes screening endoxifen in patient-derived tumor organoids. Endoxifen was tested as a single agent on tumor organoids derived from patients diagnosed with various cancer types using a personalized cancer treatment screening assay (PARIS© Test, SEngine Precision Medicine). The screen utilized patient-derived tumor organoids (PDTOs) along with high-throughput drug screening and machine learning-guided data analysis to quantify the efficacy of endoxifen against samples derived from individual patients diagnosed with various cancer types. PDTOs are self-organizing, three-dimensional organoid structures that recapitulate structural, functional, and genomic aspects of in vivo organs. The PDTOs were grown in culture from patient tumor biopsy samples.

Fresh patient biopsy samples received following surgery were cleaned and purified. Immune cells (e.g., lymphocytes, blood cells), cancer-associated fibroblasts, and other non-tumor cells were removed to establish pure PDTOs for endoxifen testing. The tumor organoids were grown and expanded by supplying organ- and patient-specific growth factors and nutrients. The PDTOs were treated with increasing concentrations of endoxifen ranging from about 30 nM to about 10 μM, and the drug response was measured using a luminescence readout. A lower luminescence was indicative of a stronger drug response.

The results were analyzed using a “SEngine Precision Medicine” (SPM) scoring system to assess the sensitivity and personalization of each tumor organoid to endoxifen. The SPM score correlates with clinical outcomes, such that a higher SPM score for a specific PDTO-drug combination predicts a more favorable clinical outcome following treatment of the corresponding patient with the tested drug.

### Example 2

### Testing Endoxifen Efficacy Against 17 Distinct Cancer Types

This example describes testing endoxifen efficacy against 17 distinct cancer types in patient-derived tumor organoids (PDTOs). Screening of PDTOs was performed as described in EXAMPLE 1. For each PDTO sample, the drug response was measured using a luminescent readout over a range of endoxifen concentrations. The drug response was evaluated based on the area under the fitted dose response curve (AUC) and the SPM scoring system. AUC values were normalized for each PDTO. Briefly, data points at each drug concentration were first normalized to a negative control, then a dose response drug curve was approximated using a four-parameter logistic model fitted to the normalized data points. The area under the curve was calculated from the normalized curve. Normalization allowed for comparison of AUC values between patient samples. The SPM scores were based on machine learning models that incorporate values for how well the drug kill patient cells (sensitivity metric), the relative drug responses of a patient to all other patients in a sample database (personalization metric), and knowledge of clinical outcomes for patients that had been evaluated with the personalized cancer screening assay (PARIS® Test, SEngine Precision Medicine).

The SPM scoring system evaluated whether a PDTO was unresponsive or responsive to endoxifen and the degree of drug response for PDTOs that were responsive to endoxifen. The SPM scoring system generated metrics an SPM score, predictive of clinical outcome, overall drug response (response category), drug sensitivity (sensitivity category), and drug personalization (personalization category). SPM scores ranged from 1-15, corresponding to the following drug response categories: no response (SPM 1-8), low response (SPM 9), moderate response (SPM 10-11), good response (SPM 12-13), and exceptional response (SPM 14-15). Drugs that exhibit exceptional responses (SPM 14-15) often indicate that a drug has clinical relevance as a monotherapy. Moderate to good drug responses (SPM 10-13) may indicate that a drug has clinical relevance, possibly in combination with other drugs. The sensitivity category was determined from the sensitivity metric and is a measure of overall cell death of a patient's cells in response to endoxifen at six concentrations. Sensitivity categories range from High, Good, Moderate, Low, to None. The personalization category was determined from the personalization metric and is a measure of how well a PDTO responded to endoxifen relative to other PDTOs evaluated using the same personalized cancer screening assay. Personalization categories range from High, Good, Moderate, Low, to None.

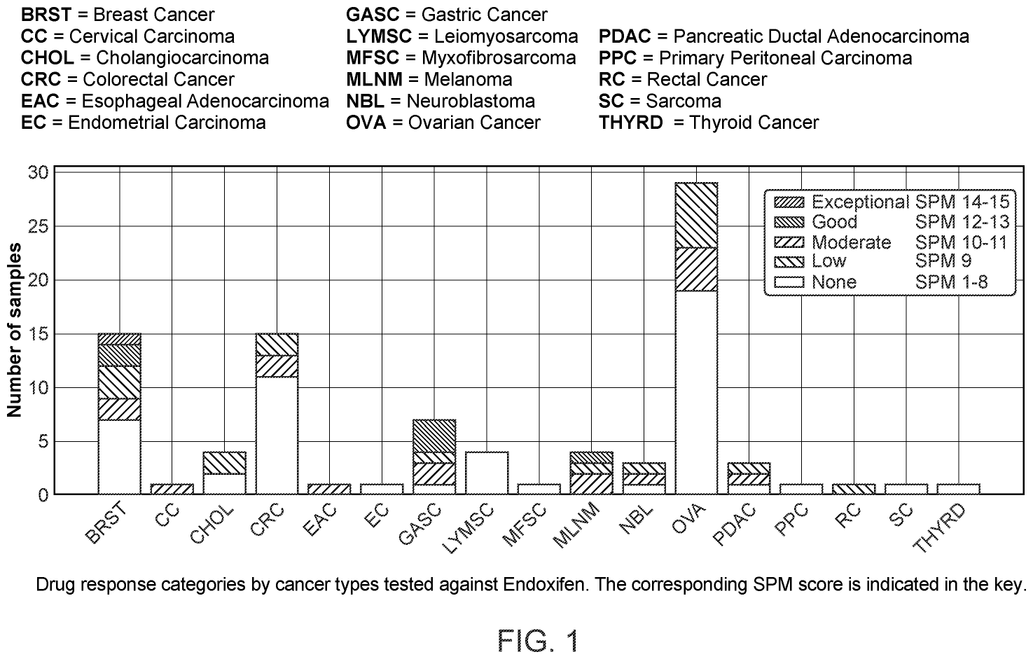

As shown in FIG. 1 and TABLE 1, Endoxifen elicited a drug response in samples from patients with breast cancer, cervical cancer, cholangiocarcinoma, colorectal cancer, esophageal adenocarcinoma, gastric cancer, melanoma, neuroblastoma, ovarian cancer, pancreatic ductal adenocarcinoma, and rectal cancer. FIG. 1 illustrates the number of samples tested for each cancer type and the drug response category for each sample. The SPM score, sensitivity category, personalization category, and response category for each sample are provided in TABLE 1. Also provided in TABLE 1 for each sample are the concentration of endoxifen that inhibits 50% of the tumor cells in vitro (IC₅₀), the AUC, and the goodness of fit (GOF) of the four-parameter logistic curve to the six experimental data points.

TABLE 1
Results of Endoxifen Screen in Patient-Derived Tumor Organoids

| | | | SPM | Sensitivity | Personalization | Response | | | |
|---|---|---|---|---|---|---|---|---|---|
| ID | Cancer Type | Diagnosis | Score | Category | Category | Category | IC50 (M) | AUC | GOF |
| 11 | Breast cancer | Triple Negative Breast Cancer | 1 | None | None | None | 9.45E−06 | 0.9911 | 0.9548 |
| 12 | Breast cancer | Triple Negative Breast Cancer | 1 | None | None | None | | 0.9378 | 0.4400 |
| 15 | Breast cancer | Triple Negative Breast Cancer | 14 | Good | High | Exceptional | 4.9E−07 | 0.4774 | 0.9417 |
| 16 | Breast cancer | Triple Negative Breast Cancer | 13 | Moderate | High | Good | 1.47E−06 | 0.5912 | 0.9449 |
| 26 | Breast cancer | HER2+ Breast Cancer | 10 | Low | Moderate | Moderate | | 0.8248 | 0.4922 |
| 36 | Breast cancer | Triple Negative Breast Cancer | 1 | None | None | None | 9.6E−06 | 0.9311 | 0.3234 |
| 45 | Breast cancer | Triple Negative Breast Cancer | 9 | Low | Low | Low | 4.22E−06 | 0.8639 | 0.9938 |
| 55 | Breast cancer | Metastatic Breast Cancer | 13 | Moderate | High | Good | 3.09E−06 | 0.6798 | 0.7984 |
| 56 | Breast cancer | Metastatic Breast Cancer | 11 | Low | Good | Moderate | 5.26E−06 | 0.8079 | 0.9547 |
| 64 | Breast cancer | T2N0 Left Breast Cancer, Stage | 4 | Low | None | None | 7.25E−06 | 0.9835 | 0.9537 |
| | | 2A | | | | | | | |
| 79 | Breast cancer | Breast Cancer | 9 | Low | Low | Low | 5.7E−06 | 0.8609 | 0.8784 |
| 80 | Breast cancer | Metastatic breast cancer | 1 | None | Low | None | 8.95E−06 | 0.8997 | 0.9762 |
| 85 | Breast cancer | Breast Cancer | 9 | Low | Low | Low | 9.04E−06 | 0.8847 | 0.9476 |
| 87 | Breast cancer | Metastatic Breast Cancer | 1 | None | None | None | | 0.9811 | 0.1883 |
| 88 | Breast cancer | Metastatic Breast Cancer | 1 | None | None | None | | 0.9320 | 0.6429 |
| 10 | Cervical carcinoma | Cervical Carcinoma | 11 | Low | Good | Moderate | 3.48E−06 | 0.8002 | 0.9956 |
| 13 | Cholangiocarcinoma | Metastatic Cholangiocarcinoma | 1 | None | None | None | 8.7E−06 | 0.9497 | 0.9935 |
| 35 | Cholangiocarcinoma | Cholangiocarcinoma | 9 | Low | Low | Low | 9.19E−06 | 0.9011 | 0.9990 |
| 62 | Cholangiocarcinoma | Cholangiocarcinoma | 3 | Low | None | None | | 0.9669 | 0.0342 |
| 9 | Colorectal cancer | Colorectal Cancer | 1 | None | None | None | 7.16E−06 | 0.9517 | 0.9996 |
| 18 | Colorectal cancer | Colorectal Cancer | 1 | None | None | None | 9.23E−06 | 0.9919 | 0.9419 |
| 23 | Colorectal cancer | Descending Colon Cancer | 1 | None | None | None | 7.23E−06 | 1.0338 | 0.9599 |
| 24 | Colorectal cancer | Colorectal Cancer | 3 | Low | None | None | 5.61E−06 | 0.9902 | 0.8804 |
| 41 | Colorectal cancer | Colorectal Cancer | 1 | None | None | None | 0.00001 | 1.0377 | 0.9329 |
| 51 | Colorectal cancer | Colorectal Cancer | 5 | Low | None | None | 5.65E−06 | 0.9305 | 0.9737 |
| 52 | Colorectal cancer | Adenocarcinoma NOS of the | 9 | Low | Low | Low | 4.72E−06 | 0.8841 | 0.9695 |
| | | Colon | | | | | | | |
| 53 | Colorectal cancer | Sigmoid Colon Cancer | 11 | Low | Good | Moderate | 3.62E−06 | 0.8215 | 0.9923 |
| 54 | Colorectal cancer | Colorectal Cancer | 1 | None | None | None | 8.86E−06 | 0.9346 | 0.9841 |
| 59 | Colorectal cancer | Colon | 10 | Low | Moderate | Moderate | 5.67E−06 | 0.8357 | 1.0000 |
| 67 | Colorectal cancer | Colorectal Cancer | 9 | Low | Low | Low | 6.74E−06 | 0.8866 | 0.9645 |
| 68 | Colorectal cancer | Colorectal Cancer | 1 | None | None | None | 9.61E−06 | 0.9755 | 0.9768 |
| 76 | Colorectal cancer | Colorectal Cancer | 1 | None | None | None | 9.09E−06 | 0.9804 | 0.9504 |
| 77 | Colorectal cancer | Stage IV Colorectal Cancer | 1 | None | None | None | | 0.9752 | 0.6094 |
| 78 | Colorectal cancer | Stage III Colorectal Cancer | 1 | None | None | None | | 1.0467 | −0.0326 |
| 39 | Endometrial | Endometrioid Adenocarcinoma | 1 | None | None | None | | 1.0102 | 0.6885 |
| | carcinoma | | | | | | | | |
| 14 | Esophageal | Metastatic Esophageal Cancer | 11 | Low | Good | Moderate | 3.97E−06 | 0.7771 | 0.9761 |
| | adenocarcinoma | | | | | | | | |
| 27 | Gastric cancer | Gastric Cancer | 5 | Low | None | None | 6.55E−06 | 0.9503 | 0.9805 |
| 28 | Gastric cancer | Gastric Cancer | 9 | Low | Low | Low | 6.73E−06 | 0.8641 | 0.9496 |
| 30 | Gastric cancer | Gastric Cancer | 12 | Low | High | Good | 2.45E−06 | 0.7529 | 0.9786 |
| 31 | Gastric cancer | Gastric Cancer | 11 | Low | Good | Moderate | 4.14E−06 | 0.8116 | 0.9757 |
| 47 | Gastric cancer | Gastric Cancer | 13 | Moderate | High | Good | 2.89E−06 | 0.6723 | 0.9950 |
| 48 | Gastric cancer | Gastric Cancer | 10 | Low | Moderate | Moderate | 4.86E−06 | 0.8547 | 0.9802 |
| 17 | Leiomyosarcoma | Metastatic Uterine | 1 | None | None | None | | 0.9719 | 0.5832 |
| | | Leiomyosarcoma | | | | | | | |
| 37 | Leiomyosarcoma | Uterine Leiomyosarcoma | 1 | None | None | None | | 1.0448 | 0.2352 |
| 40 | Leiomyosarcoma | Leiomyosarcoma | 1 | None | None | None | | 1.0001 | 0.1150 |
| 60 | Melanoma | Melanoma, Anorectal | 9 | Low | Low | Low | 2.74E−06 | 0.8952 | 0.9153 |
| 61 | Melanoma | Acral Melanoma | 11 | Low | Good | Moderate | 3.3E−06 | 0.7865 | 0.9906 |
| 66 | Melanoma | Acral Melanoma | 13 | Moderate | High | Good | 1.44E−06 | 0.6541 | 0.9861 |
| 84 | Melanoma | Recurrent Acral Melanoma | 11 | Low | Good | Moderate | 7.06E−06 | 0.8094 | 0.9087 |
| 1 | Myxofibrosarcoma | Myxofibrosarcoma | 1 | None | None | None | 7.25E−06 | 0.9384 | 0.9839 |
| 3 | Neuroblastoma | Neuroblastoma | 9 | Low | Low | Low | 5.73E−06 | 0.8848 | 0.9623 |
| 6 | Neuroblastoma | Neuroblastoma | 10 | Low | Moderate | Moderate | 5.87E−06 | 0.8184 | 0.9951 |
| 7 | Neuroblastoma | Neuroblastoma | 1 | None | None | None | 7.18E−06 | 0.9511 | 0.9575 |
| 2 | Ovarian cancer | Malignant Endometrioid Ovarian | 1 | None | Low | None | 4.87E−06 | 0.9081 | 0.9767 |
| | | Carcinoma | | | | | | | |
| 5 | Ovarian cancer | Ovarian Cancer | 1 | None | None | None | 7.26E−06 | 0.9209 | 0.9961 |
| 20 | Ovarian cancer | Clear Cell Ovarian Carcinoma | 1 | None | None | None | 9.3E−06 | 1.0105 | 0.9088 |
| 25 | Ovarian cancer | Stage IIIC Poorly-Differentiated | 9 | Low | Low | Low | 3.08E−06 | 0.8454 | 0.9594 |
| | | Serous Adenocarcinoma to the | | | | | | | |
| | | Ovary | | | | | | | |
| 29 | Ovarian cancer | Fallopian Tube Carcinoma | 9 | Low | Low | Low | 6.91E−06 | 0.8909 | 0.9086 |
| 32 | Ovarian cancer | Ovarian Cancer | 5 | Low | None | None | | 0.9267 | 0.3813 |
| 33 | Ovarian cancer | Stage IV Ovarian Cancer | 1 | None | None | None | 9.33E−06 | 0.9074 | 0.9081 |
| 34 | Ovarian cancer | Ovarian | 10 | Low | Moderate | Moderate | 3.45E−06 | 0.8343 | 0.5737 |
| 38 | Ovarian cancer | Ovarian Cancer | 1 | None | None | None | 9.41E−06 | 0.9838 | 0.7887 |
| 42 | Ovarian cancer | Granulosa Cell Tumor of Ovary | 10 | Low | Moderate | Moderate | 6.22E−06 | 0.8320 | 0.9037 |
| 43 | Ovarian cancer | Unknown - Bilateral Adrenal | 1 | None | None | None | | 1.0057 | 0.3869 |
| | | Masses; Carcinomatosis | | | | | | | |
| 44 | Ovarian cancer | High Grade Carcinoma of | 1 | None | None | None | | 0.9637 | 0.9400 |
| | | Mullerian/Ovarian Origin | | | | | | | |
| 46 | Ovarian cancer | Platinum-Resistant Recurrent | 1 | None | None | None | 9.47E−06 | 1.1417 | 0.7864 |
| | | Ovarian Cancer | | | | | | | |
| 49 | Ovarian cancer | High Grade Serous Carcinoma of | 1 | None | None | None | 6.17E−06 | 1.0287 | 0.9424 |
| | | Ovary | | | | | | | |
| 50 | Ovarian cancer | Ovarian | 9 | Low | Low | Low | 7.21E−06 | 0.8785 | 0.8962 |
| 57 | Ovarian cancer | Ovarian Cancer | 9 | Low | Low | Low | 6.13E−06 | 0.8853 | 0.9096 |
| 58 | Ovarian cancer | Fallopian Tube Cancer | 1 | None | None | None | 4.14E−06 | 0.9968 | 0.9401 |
| 63 | Ovarian cancer | Ovarian | 5 | Low | None | None | 6.06E−06 | 0.9220 | 0.9922 |
| 65 | Ovarian cancer | Ovarian Cancer | 11 | Low | Good | Moderate | 3.59E−06 | 0.8174 | 0.8005 |
| 69 | Ovarian cancer | Ovarian Mucinous | 4 | Low | None | None | | 0.9760 | 0.9734 |
| | | Adenocarcinoma | | | | | | | |
| 70 | Ovarian cancer | Ovarian Cancer | 9 | Low | Low | Low | 5.48E−06 | 0.8739 | 0.9830 |
| 71 | Ovarian cancer | Metastatic Ovarian cancer | 9 | Low | Low | Low | 3.69E−06 | 0.8607 | 0.9242 |
| 73 | Ovarian cancer | High Grade Serous Carcinoma | 1 | None | None | None | 4.28E−06 | 1.0172 | 0.9260 |
| | | consistent with a Mullerian | | | | | | | |
| | | Primary | | | | | | | |
| 74 | Ovarian cancer | Stage IV Ovarian Cancer, | 1 | None | None | None | 9.16E−06 | 0.9895 | 0.9372 |
| | | Mullerian Origin | | | | | | | |
| 75 | Ovarian cancer | Low Grade Serous Ovarian Cancer | 1 | None | None | None | | 0.9934 | 0.9087 |
| 82 | Ovarian cancer | Ovarian | 1 | None | None | None | 9.26E−06 | 0.9109 | 0.9251 |
| 83 | Ovarian cancer | Ovarian Cancer | 1 | None | None | None | | 1.0762 | 0.4356 |
| 86 | Ovarian cancer | Recurrent Ovarian Cancer, | 11 | Low | Good | Moderate | 8.13E−06 | 0.8038 | 0.9483 |
| | | Peritoneal Carcinomatosis | | | | | | | |
| 89 | Ovarian cancer | Ovarian Cancer | 5 | Low | None | None | 6.7E−06 | 0.9291 | 0.9815 |
| 8 | Pancreatic ductal | Pancreatic Cancer | 11 | Low | Good | Moderate | 4.47E−06 | 0.8283 | 0.9910 |
| | adenocarcinoma | | | | | | | | |
| 22 | Pancreatic ductal | Metastatic Pancreatic Ductal | 9 | Low | Low | Low | 5.83E−06 | 0.8839 | 0.9535 |
| | adenocarcinoma | Adenocarcinoma, Stage III | | | | | | | |
| 81 | Pancreatic ductal | Pancreatic Ductal Adenocarcinoma | 1 | None | None | None | | 1.1358 | 0.7478 |
| | adenocarcinoma | | | | | | | | |
| 72 | Primary Peritoneal | Stage IV Primary Peritoneal | 1 | None | None | None | 9.37E−06 | 0.9623 | 0.9839 |
| | Carcinoma | Carcinoma | | | | | | | |
| 19 | Rectal cancer | Rectum adenocarcinoma | 9 | Low | Low | Low | 3.9E−06 | 0.8614 | 0.9757 |
| 21 | Sarcoma | Epitheloid Hemangioendothelioma | 1 | None | None | None | | 0.9995 | 0.4631 |
| 4 | Thyroid cancer | Papillary Thyroid Carcinoma | 1 | None | None | None | 7.1E−06 | 0.9109 | 0.9917 |

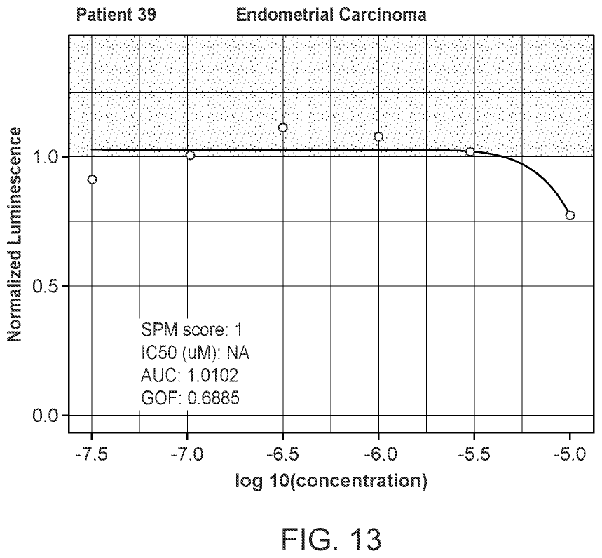

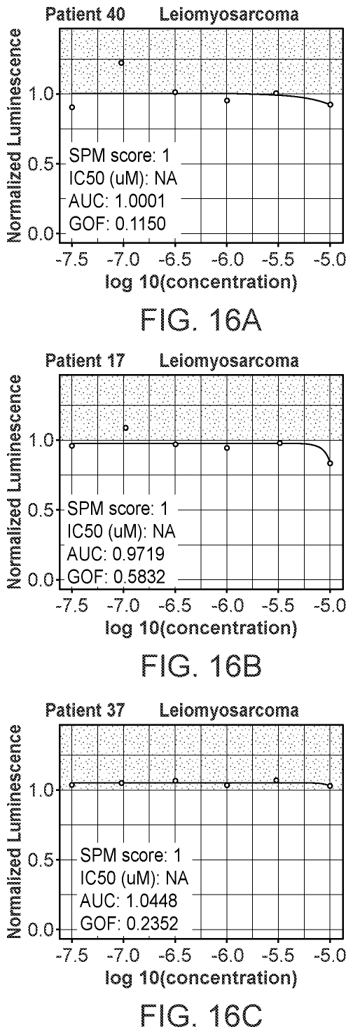

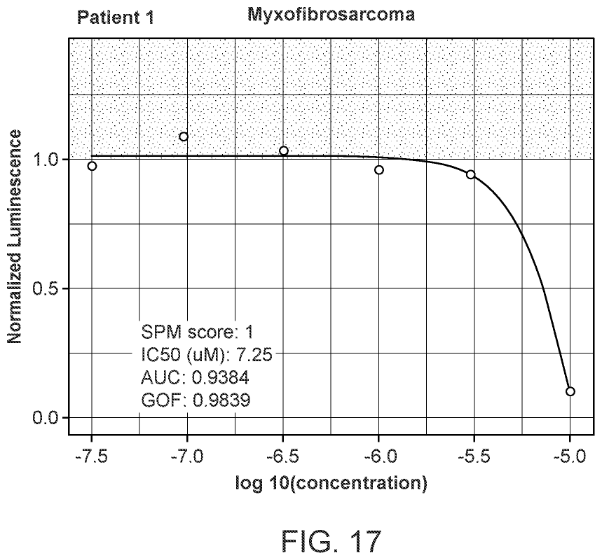

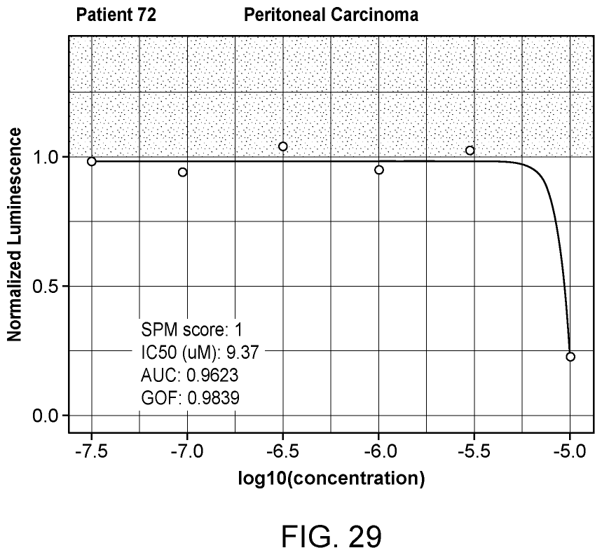

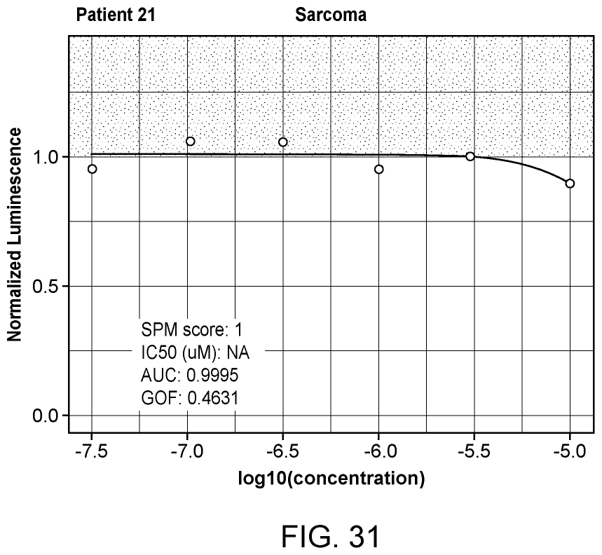

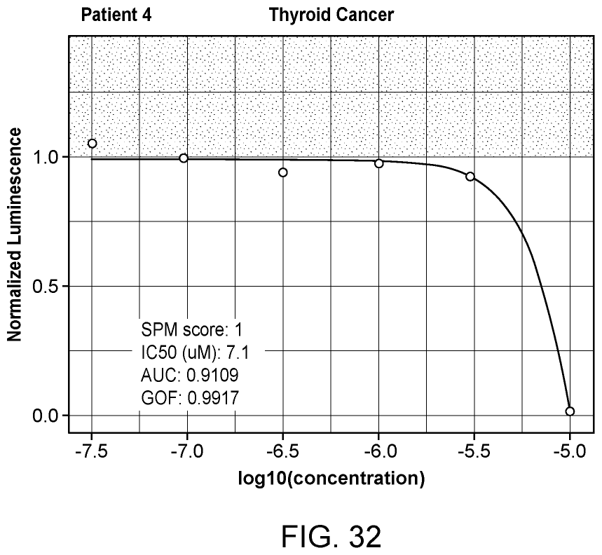

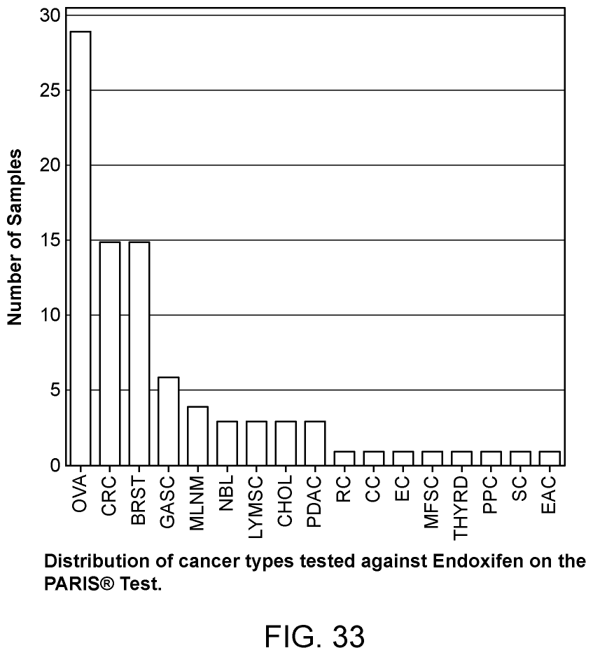

The number of patient samples tested for each cancer type is summarized in FIG. 33. Patient-specific results for individual cancer types are described in greater detail in EXAMPLE 3-EXAMPLE 13. No response was observed in the tested samples from patients with endometrial carcinoma (FIG. 13), leiomyosarcoma (FIG. 16A, FIG. 16B, and FIG. 16C), myxofibrosarcoma (FIG. 17), primary peritoneal carcinoma (FIG. 29), sarcoma (FIG. 31), or thyroid cancer (FIG. 32). However, the sample sizes for these cancer types were too low to exclude the possibility of observing a drug response to endoxifen.

### Example 3

### Breast Cancer Response to Endoxifen in Patient-Derived Samples

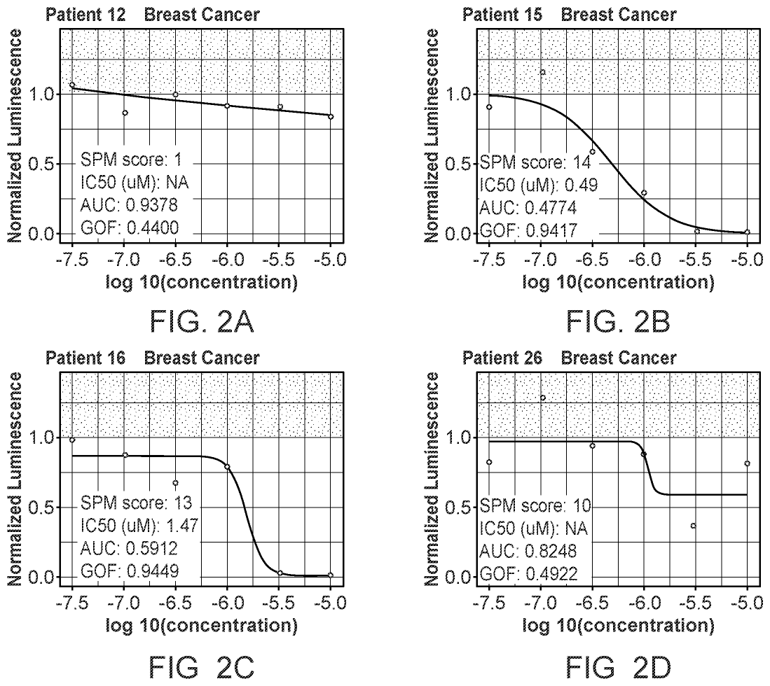

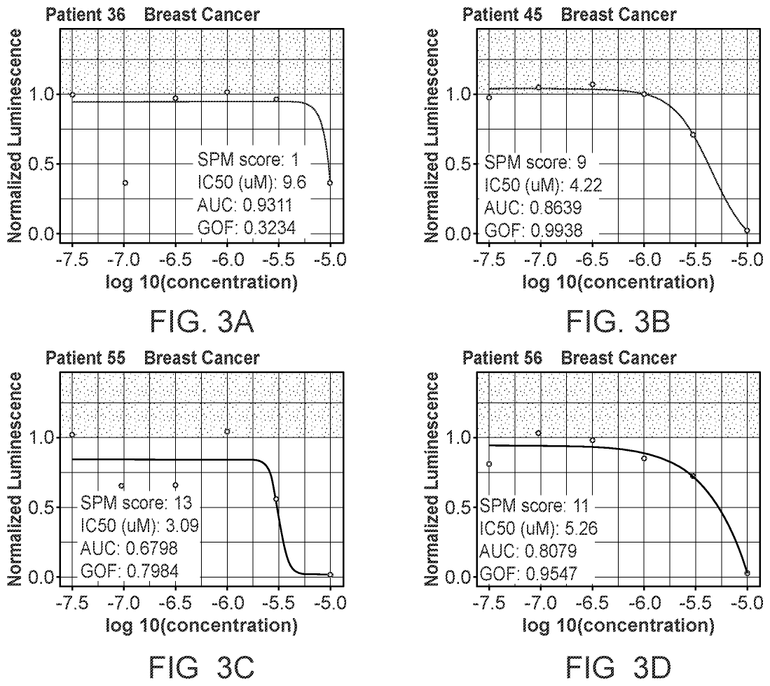

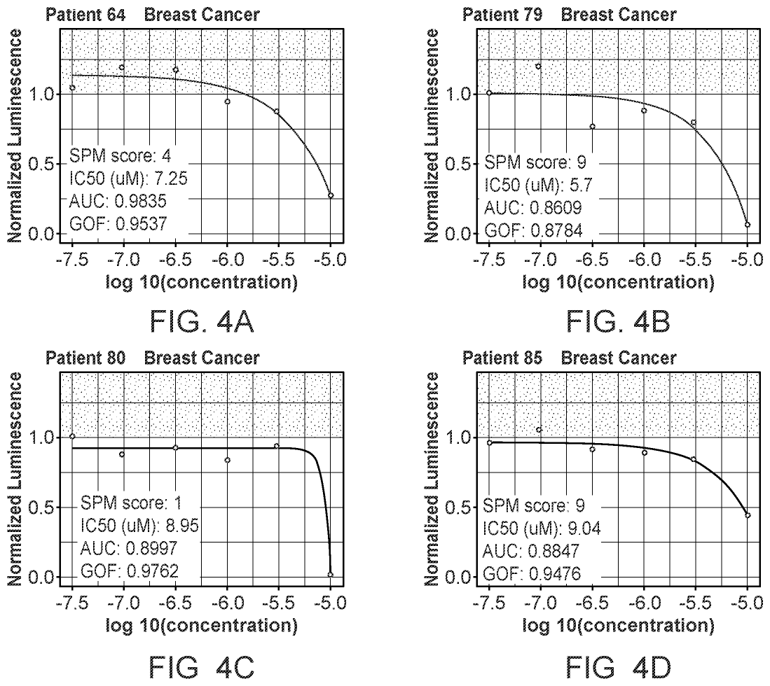

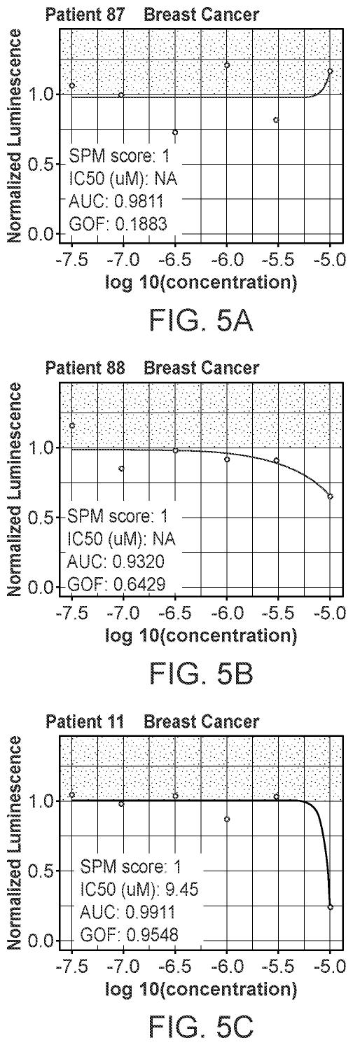

This example describes a breast cancer response to endoxifen measured in patient-derived tumor organoids (PDTOs). Screening of PDTOs was performed as described in EXAMPLE 1. PDTOs derived from 15 different breast cancer patients were screened for a response to endoxifen. Individual dose-response curves for each patient are shown in FIG. 2A, FIG. 2B, FIG. 2C, FIG. 2D, FIG. 3A, FIG. 3B, FIG. 3C, FIG. 3D, FIG. 4A, FIG. 4B, FIG. 4C, FIG. 4D, FIG. 5A, FIG. 5B, and FIG. 5C. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each breast cancer patient, along with the sensitivity, personalization, and response categories of the PDTO endoxifen response, are summarized in TABLE 2. Additional details for each patient sample are provided in TABLE 1 and EXAMPLE 2.

TABLE 2
Summary of Breast Cancer Screening Results

| | | | Personal- | |
|---|---|---|---|---|
| | | Sensitivity | ization | Response |
| ID | Diagnosis | Category | Category | Category |
| 11 | Triple Negative Breast Cancer | None | None | None |
| 12 | Triple Negative Breast Cancer | None | None | None |
| 15 | Triple Negative Breast Cancer | Good | High | Exceptional |
| 16 | Triple Negative Breast Cancer | Moderate | High | Good |
| 26 | HER2 + Breast Cancer | Low | Moderate | Moderate |
| 36 | Triple Negative Breast Cancer | None | None | None |
| 45 | Triple Negative Breast Cancer | Low | Low | Low |
| 55 | Metastatic Breast Cancer | Moderate | High | Good |
| 56 | Metastatic Breast Cancer | Low | Good | Moderate |
| 64 | T2N0 Left Breast Cancer, | Low | None | None |
| | Stage 2A | | | |
| 79 | Breast Cancer | Low | Low | Low |
| 80 | Metastatic breast cancer | None | Low | None |
| 85 | Breast Cancer | Low | Low | Low |
| 87 | Metastatic Breast Cancer | None | None | None |
| 88 | Metastatic Breast Cancer | None | None | None |

One breast cancer patient sample was categorized as having an exceptional response to endoxifen, two samples were categorized as having a good response, two samples were categorized as having a moderate response, and three samples were categorized as having a low response. Notably, of the samples from patients diagnosed with triple negative breast cancer (patients 11, 12, 15, 16, 36, and 45), one was categorized as having an exceptional response, one was categorized as having a good response, and one was categorized as having a low response. Together, these data suggest that endoxifen may be an effective treatment for various breast cancers, including triple negative breast cancer.

### Example 4

### Cervical Cancer Response to Endoxifen in Patient-Derived Samples

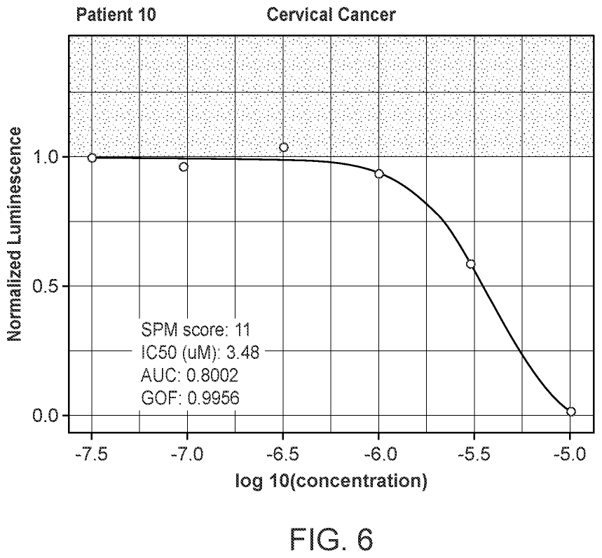

This example describes a cervical cancer response to endoxifen measured in patient-derived tumor organoids (PDTOs). Screening of PDTOs was performed as described in EXAMPLE 1. PDTOs derived from a cervical cancer patient (Patient 10) were screened for a response to endoxifen. The dose-response curve is shown in FIG. 6. Curve metrics such as the area under the curve (AUC) and goodness of fit (GOF) are provided. The IC₅₀ (μM) was calculated by a four-parameter logistic model. The patient had been diagnosed with cervical carcinoma. The sensitivity of the endoxifen response was categorized as low, the personalization of the response was categorized as good, and the response was categorized as moderate. Additional details the patient sample are provided in TABLE 1 and EXAMPLE 2. This data suggests that endoxifen may be an effective treatment for cervical cancer.

### Example 5

### Cholangiocarcinoma Response to Endoxifen in Patient-Derived Samples

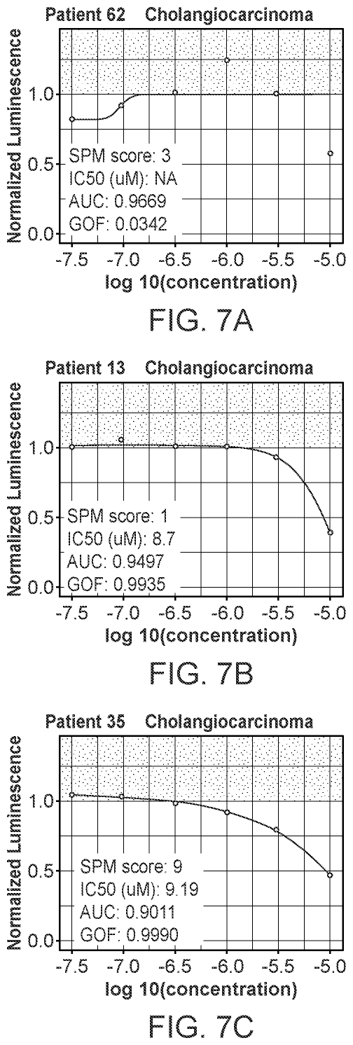

This example describes a cholangiocarcinoma response to endoxifen measured in patient-derived tumor organoids (PDTOs). Screening of PDTOs was performed as described in EXAMPLE 1. PDTOs derived from three different cholangiocarcinoma patients were screened for a response to endoxifen. Individual dose-response curves for each patient are shown in FIG. 7A, FIG. 7B, and FIG. 7C. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each cholangiocarcinoma patient, along with the sensitivity, personalization, and response categories of the PDTO endoxifen response, are summarized in TABLE 3. Additional details for each patient sample are provided in TABLE 1 and EXAMPLE 2.

TABLE 3
Summary of Cholangiocarcinoma Screening Results

| | | Sensitivity | Personalization | Response |
|---|---|---|---|---|
| ID | Diagnosis | Category | Category | Category |
| 13 | Metastatic | None | None | None |
| | Cholangiocarcinoma | | | |
| 35 | Cholangiocarcinoma | Low | Low | Low |
| 62 | Cholangiocarcinoma | Low | None | None |

One cholangiocarcinoma patient sample was categorized as having a low response to endoxifen. This data suggests that endoxifen may be an effective treatment for cholangiocarcinoma.

### Example 6

### Colorectal Cancer Response to Endoxifen in Patient-Derived Samples

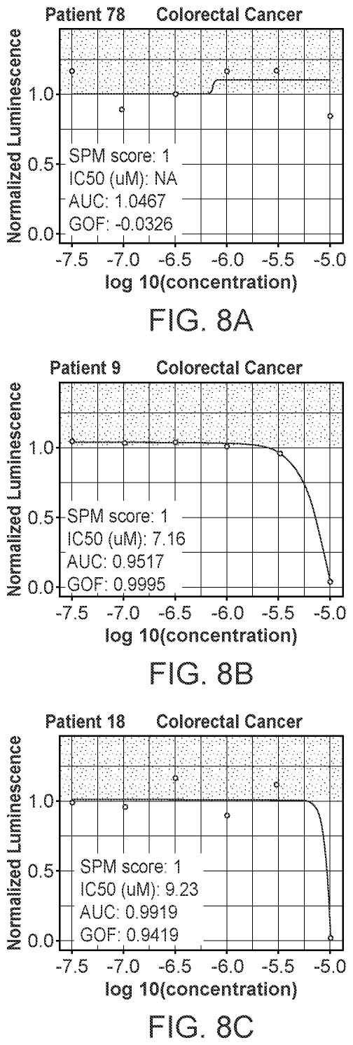

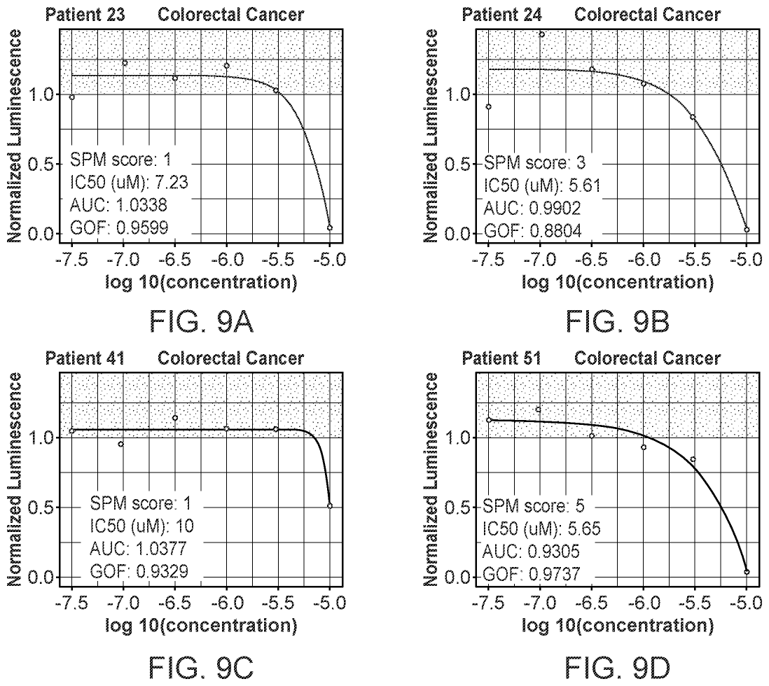

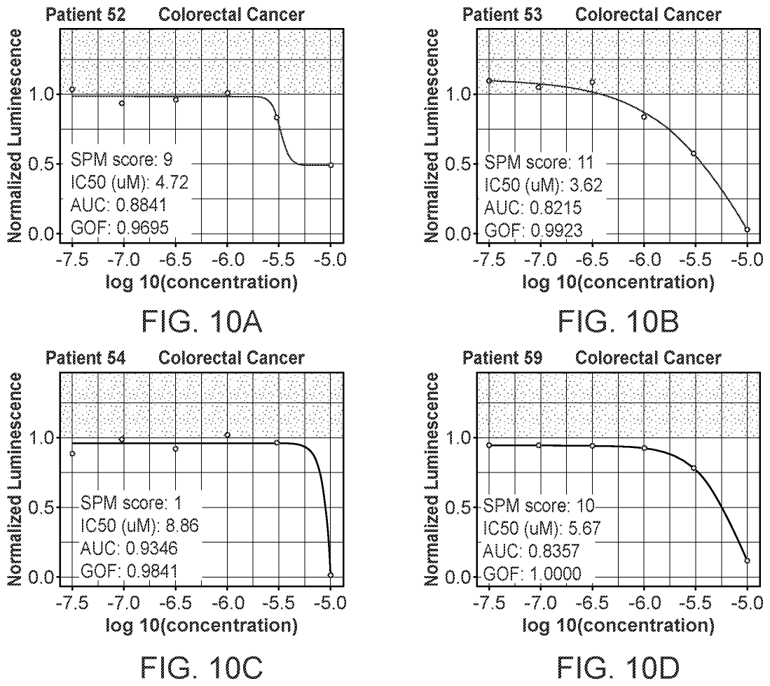

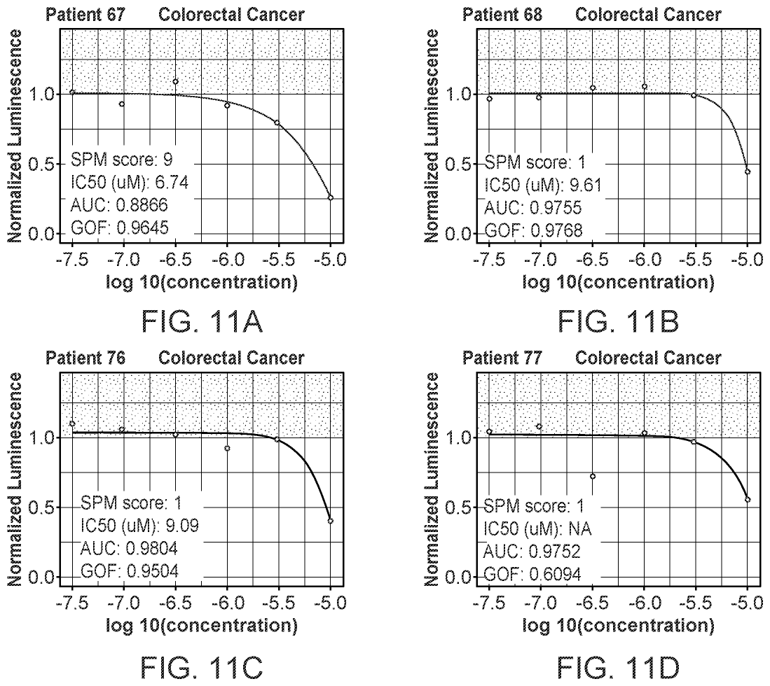

This example describes a colorectal cancer response to endoxifen measured in patient-derived tumor organoids (PDTOs). Screening of PDTOs was performed as described in EXAMPLE 1. PDTOs derived from 15 different colorectal cancer patients were screened for a response to endoxifen. Individual dose-response curves for each patient are shown in FIG. 8A, FIG. 8B, FIG. 8C, FIG. 9A, FIG. 9B, FIG. 9C, FIG. 9D, FIG. 10A, FIG. 10B, FIG. 10C, FIG. 10D, FIG. 11A, FIG. 11B, FIG. 11C, and FIG. 11D. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each colorectal cancer patient, along with the sensitivity, personalization, and response categories of the PDTO endoxifen response, are summarized in TABLE 4. Additional details for each patient sample are provided in TABLE 1 and EXAMPLE 2.

TABLE 4
Summary of Colorectal Cancer Screening Results

| | | Sensitivity | Personalization | Response |
|---|---|---|---|---|
| ID | Diagnosis | Category | Category | Category |
| 9 | Colorectal Cancer | None | None | None |
| 18 | Colorectal Cancer | None | None | None |
| 23 | Descending Colon Cancer | None | None | None |
| 24 | Colorectal Cancer | Low | None | None |
| 41 | Colorectal Cancer | None | None | None |
| 51 | Colorectal Cancer | Low | None | None |
| 52 | Adenocarcinoma NOS | Low | Low | Low |
| | of the Colon | | | |
| 53 | Sigmoid Colon Cancer | Low | Good | Moderate |
| 54 | Colorectal Cancer | None | None | None |
| 59 | Colon | Low | Moderate | Moderate |
| 67 | Colorectal Cancer | Low | Low | Low |
| 68 | Colorectal Cancer | None | None | None |
| 76 | Colorectal Cancer | None | None | None |
| 77 | Stage IV Colorectal Cancer | None | None | None |
| 78 | Stage III Colorectal Cancer | None | None | None |

Two colorectal cancer patient samples were categorized as having a moderate response to endoxifen, and two samples were categorized as having a low response. Together, these data suggest that endoxifen may be an effective treatment for colorectal cancer.

### Example 7

### Esophageal Cancer Response to Endoxifen in Patient-Derived Samples

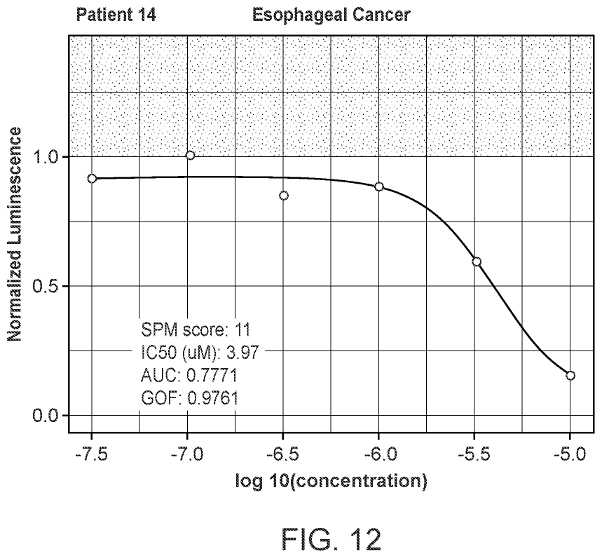

This example describes an esophageal cancer response to endoxifen measured in patient-derived tumor organoids (PDTOs). Screening of PDTOs was performed as described in EXAMPLE 1. PDTOs derived from an esophageal adenocarcinoma patient (Patient 14) were screened for a response to endoxifen. The dose-response curve is shown in FIG. 12. Curve metrics such as the area under the curve (AUC) and goodness of fit (GOF) are provided. The IC₅₀ (μM) was calculated by a four-parameter logistic model. The patient had been diagnosed with metastatic esophageal cancer. The sensitivity of the endoxifen response was categorized as low, the personalization of the response was categorized as good, and the response was categorized as moderate. Additional details the patient sample are provided in TABLE 1 and EXAMPLE 2. This data suggests that endoxifen may be an effective treatment for esophageal adenocarcinoma.

### Example 8

### Gastric Cancer Response to Endoxifen in Patient-Derived Samples

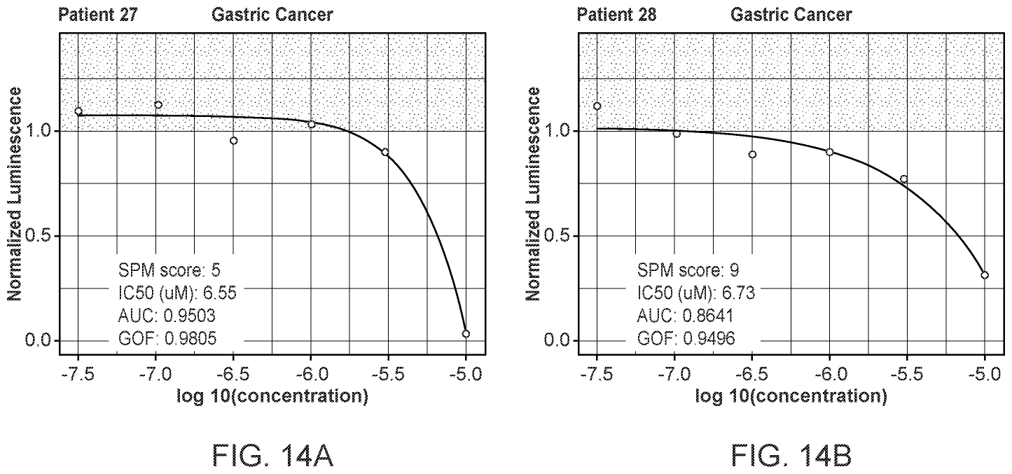

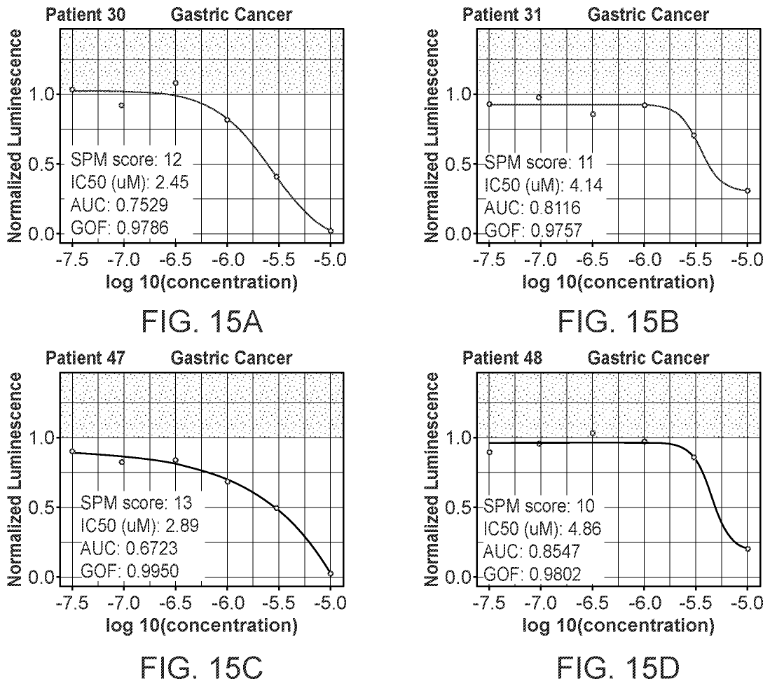

This example describes a gastric cancer response to endoxifen measured in patient-derived tumor organoids (PDTOs). Screening of PDTOs was performed as described in EXAMPLE 1. PDTOs derived from six different gastric cancer patients were screened for a response to endoxifen. Individual dose-response curves for each patient are shown in FIG. 14A, FIG. 14B, FIG. 15A, FIG. 15B, FIG. 15C, and FIG. 15D. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each gastric cancer patient, along with the sensitivity, personalization, and response categories of the PDTO endoxifen response, are summarized in TABLE 5. Additional details for each patient sample are provided in TABLE 1 and EXAMPLE 2.

TABLE 5
Summary of Gastric Cancer Screening Results

| | | Sensitivity | Personalization | Response |
|---|---|---|---|---|
| ID | Diagnosis | Category | Category | Category |
| 27 | Gastric Cancer | Low | None | None |
| 28 | Gastric Cancer | Low | Low | Low |
| 30 | Gastric Cancer | Low | High | Good |
| 31 | Gastric Cancer | Low | Good | Moderate |
| 47 | Gastric Cancer | Moderate | High | Good |
| 48 | Gastric Cancer | Low | Moderate | Moderate |

Two gastric cancer patient samples were categorized as having a good response to endoxifen, two samples were categorized as having a moderate response, and one sample was categorized as having a low response. Together, these data suggest that endoxifen may be an effective treatment for gastric cancer.

### Example 9

### Melanoma Response to Endoxifen in Patient-Derived Samples

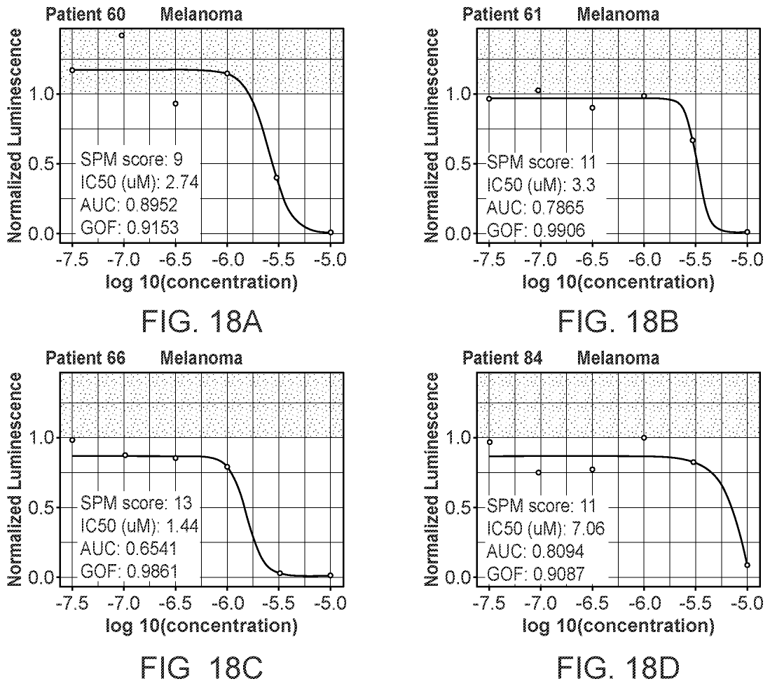

This example describes a melanoma response to endoxifen measured in patient-derived tumor organoids (PDTOs). Screening of PDTOs was performed as described in EXAMPLE 1. PDTOs derived from four different melanoma patients were screened for a response to endoxifen. Individual dose-response curves for each patient are shown in FIG. 18A, FIG. 18B, FIG. 18C, and FIG. 18D. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each melanoma patient, along with the sensitivity, personalization, and response categories of the PDTO endoxifen response, are summarized in TABLE 6. Additional details for each patient sample are provided in TABLE 1 and EXAMPLE 2.

TABLE 6
Summary of Melanoma Screening Results

| | | Sensitivity | Personalization | Response |
|---|---|---|---|---|
| ID | Diagnosis | Category | Category | Category |
| 60 | Melanoma, Anorectal | Low | Low | Low |
| 61 | Acral Melanoma | Low | Good | Moderate |
| 66 | Acral Melanoma | Moderate | High | Good |
| 84 | Recurrent Acral Melanoma | Low | Good | Moderate |

One melanoma patient sample was categorized as having a good response to endoxifen, two samples were categorized as having a moderate response, and one sample was categorized as having a low response. Together, these data suggest that endoxifen may be an effective treatment for melanoma.

### Example 10

### Neuroblastoma Response to Endoxifen in Patient-Derived Samples

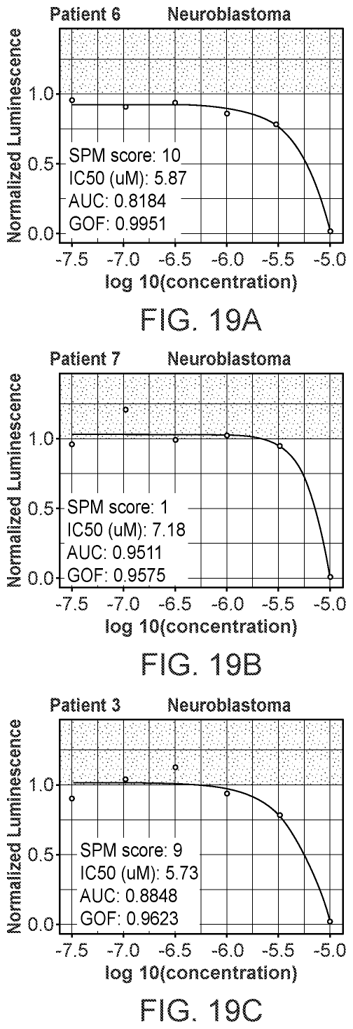

This example describes a neuroblastoma response to endoxifen measured in patient-derived tumor organoids (PDTOs). Screening of PDTOs was performed as described in EXAMPLE 1. PDTOs derived from three different neuroblastoma patients were screened for a response to endoxifen. Individual dose-response curves for each patient are shown in FIG. 19A, FIG. 19B, and FIG. 19C. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each neuroblastoma patient, along with the sensitivity, personalization, and response categories of the PDTO endoxifen response, are summarized in TABLE 7. Additional details for each patient sample are provided in TABLE 1 and EXAMPLE 2.

TABLE 7
Summary of Neuroblastoma Screening Results

| | | Sensitivity | Personalization | Response |
|---|---|---|---|---|
| ID | Diagnosis | Category | Category | Category |
| 3 | Neuroblastoma | Low | Low | Low |
| 6 | Neuroblastoma | Low | Moderate | Moderate |
| 7 | Neuroblastoma | None | None | None |

One neuroblastoma patient sample was categorized as having a moderate response to endoxifen, and one sample was categorized as having a low response. Together, these data suggest that endoxifen may be an effective treatment for neuroblastoma.

### Example 11

### Ovarian Cancer Response to Endoxifen in Patient-Derived Samples

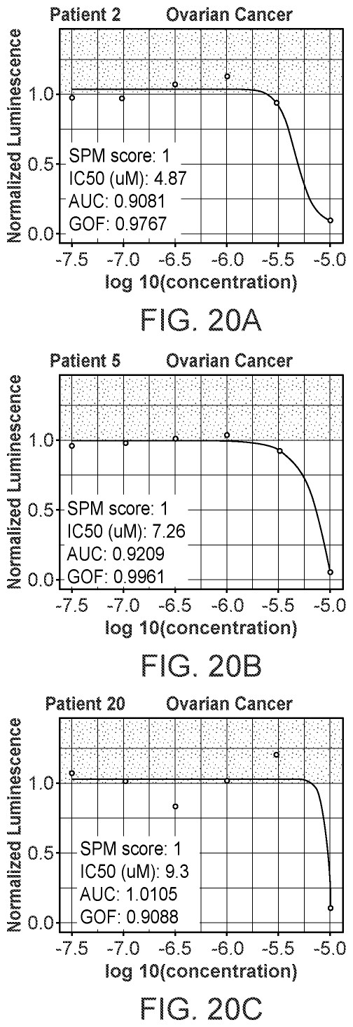

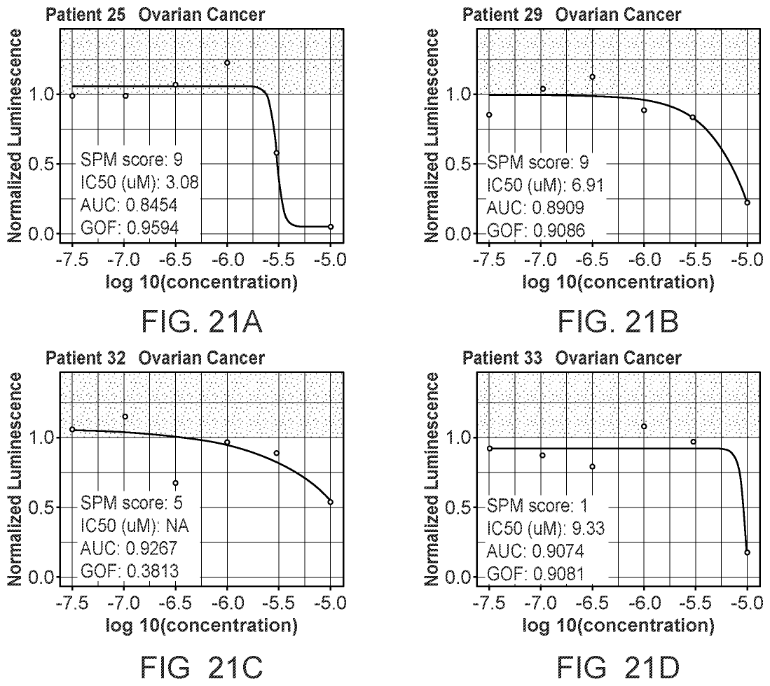

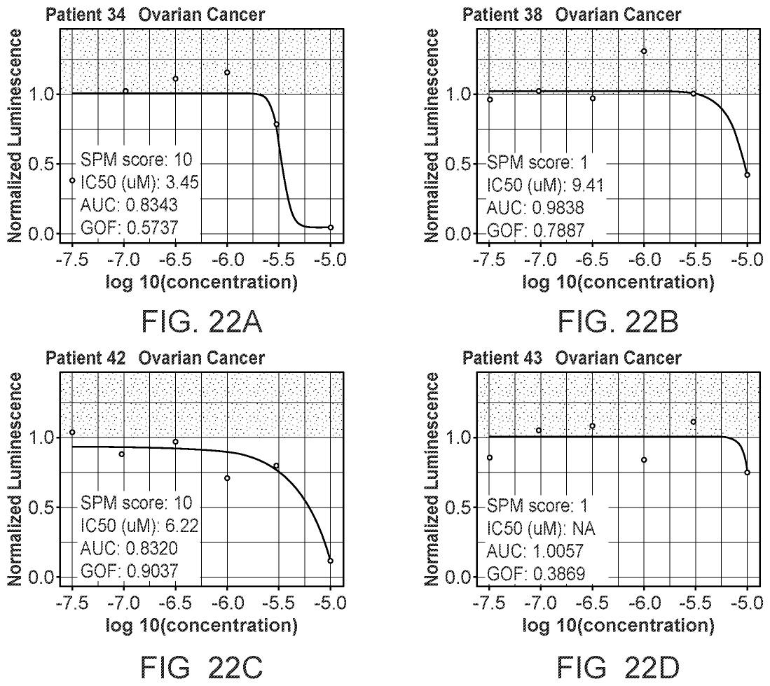

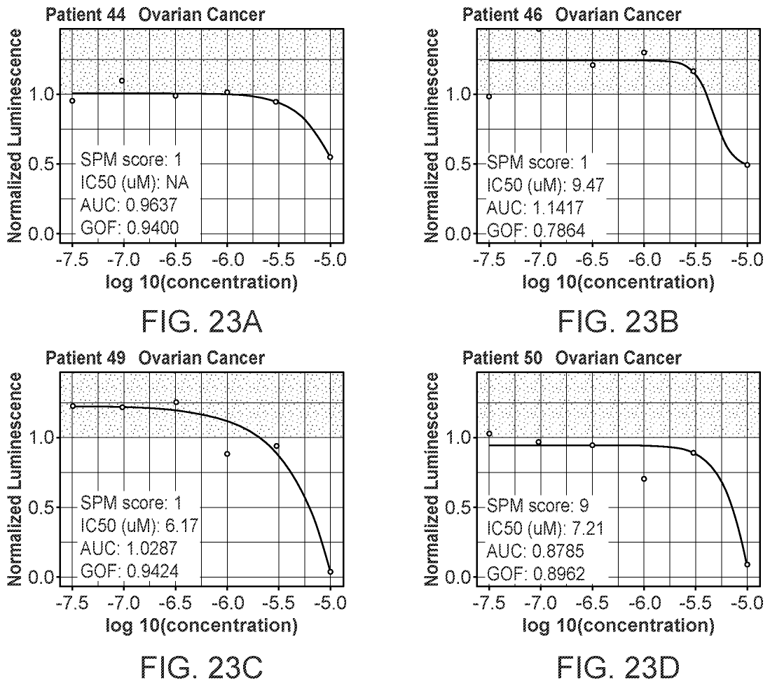

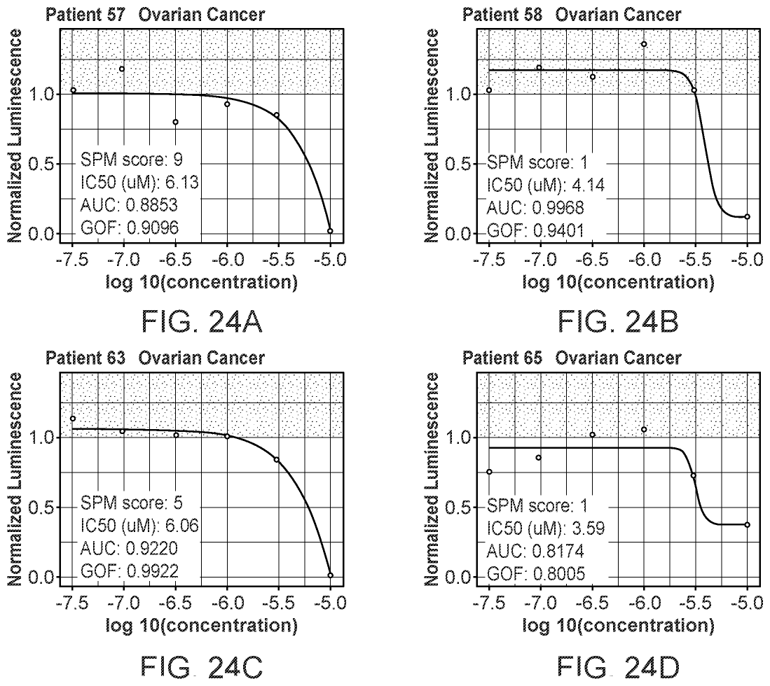

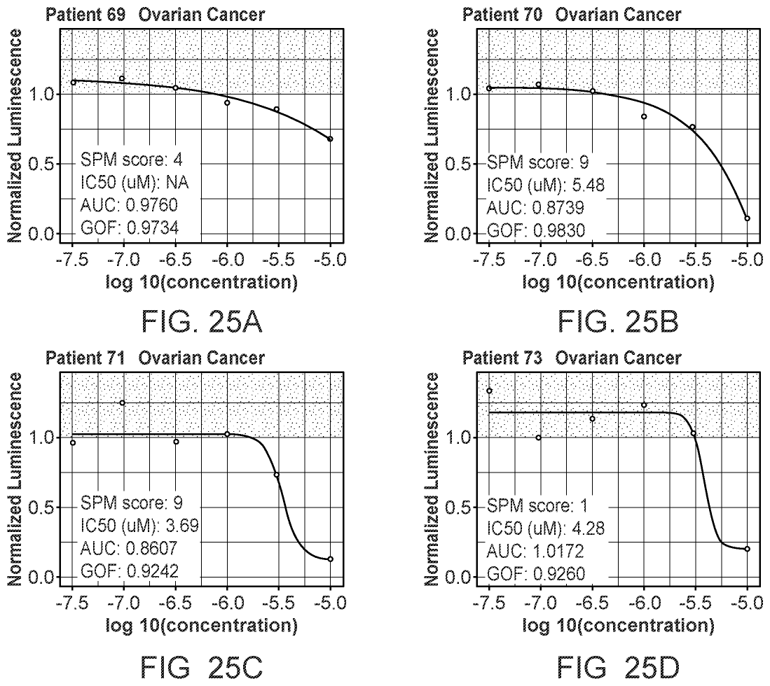

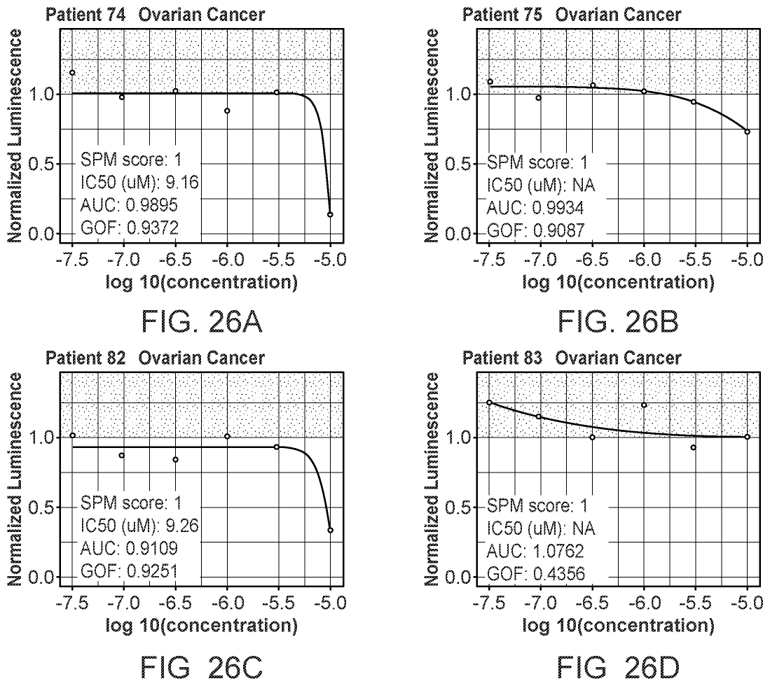

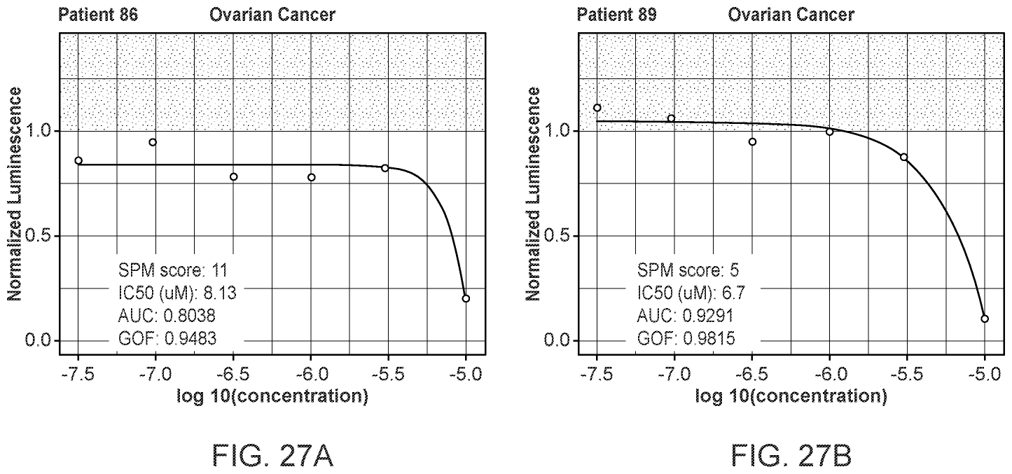

This example describes an ovarian cancer response to endoxifen measured in patient-derived tumor organoids (PDTOs). Screening of PDTOs was performed as described in EXAMPLE 1. PDTOs derived from 29 different ovarian cancer patients were screened for a response to endoxifen. Individual dose-response curves for each patient are shown in FIG. 20A, FIG. 20B, FIG. 20C, FIG. 21A, FIG. 21B, FIG. 21C, FIG. 21D, FIG. 22A, FIG. 22B, FIG. 22C, FIG. 22D, FIG. 23A, FIG. 23B, FIG. 23C, FIG. 23D, FIG. 24A, FIG. 24B, FIG. 24C, FIG. 24D, FIG. 25A, FIG. 25B, FIG. 25C, FIG. 25D, FIG. 26A, FIG. 26B, FIG. 26C, FIG. 26D, FIG. 27A, and FIG. 27B. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each ovarian cancer patient, along with the sensitivity, personalization, and response categories of the PDTO endoxifen response, are summarized in TABLE 8. Additional details for each patient sample are provided in TABLE 1 and EXAMPLE 2.

TABLE 8
Summary of Ovarian Cancer Screening Results

| | | | Personal- | |
|---|---|---|---|---|
| | | Sensitivity | ization | Response |
| ID | Diagnosis | Category | Category | Category |
| 2 | Malignant Endometrioid | None | Low | None |
| | Ovarian Carcinoma | | | |
| 5 | Ovarian Cancer | None | None | None |
| 20 | Clear Cell Ovarian Carcinoma | None | None | None |
| 25 | Stage IIIC Poorly-Differentiated | Low | Low | Low |
| | Serous Adenocarcinoma to the | | | |
| | Ovary | | | |
| 29 | Fallopian Tube Carcinoma | Low | Low | Low |
| 32 | Ovarian Cancer | Low | None | None |
| 33 | Stage IV Ovarian Cancer | None | None | None |
| 34 | Ovarian | Low | Moderate | Moderate |
| 38 | Ovarian Cancer | None | None | None |
| 42 | Granulosa Cell Tumor of Ovary | Low | Moderate | Moderate |
| 43 | Unknown - Bilateral Adrenal | None | None | None |
| | Masses; Carcinomatosis | | | |
| 44 | High Grade Carcinoma of | None | None | None |
| | Mullerian/Ovarian Origin | | | |
| 46 | Platinum-Resistant Recurrent | None | None | None |
| | Ovarian Cancer | | | |
| 49 | High Grade Serous Carcinoma | None | None | None |
| | of Ovary | | | |
| 50 | Ovarian | Low | Low | Low |
| 57 | Ovarian Cancer | Low | Low | Low |
| 58 | Fallopian Tube Cancer | None | None | None |
| 63 | Ovarian | Low | None | None |
| 65 | Ovarian Cancer | Low | Good | Moderate |
| 69 | Ovarian Mucinous | Low | None | None |
| | Adenocarcinoma | | | |
| 70 | Ovarian Cancer | Low | Low | Low |
| 71 | Metastatic Ovarian cancer | Low | Low | Low |
| 73 | High Grade Serous Carcinoma | None | None | None |
| | consistent with a Mullerian | | | |
| | Primary | | | |
| 74 | Stage IV Ovarian Cancer, | None | None | None |
| | Mullerian Origin | | | |
| 75 | Low Grade Serous Ovarian | None | None | None |
| | Cancer | | | |
| 82 | Ovarian | None | None | None |
| 83 | Ovarian Cancer | None | None | None |
| 86 | Recurrent Ovarian Cancer, | Low | Good | Moderate |
| | Peritoneal Carcinomatosis | | | |
| 89 | Ovarian Cancer | Low | None | None |

Four ovarian cancer patient sample was categorized as having a moderate response to endoxifen, and six samples were categorized as having a low response. Together, these data suggest that endoxifen may be an effective treatment for ovarian cancer.

### Example 12

### Pancreatic Cancer Response to Endoxifen in Patient-Derived Samples

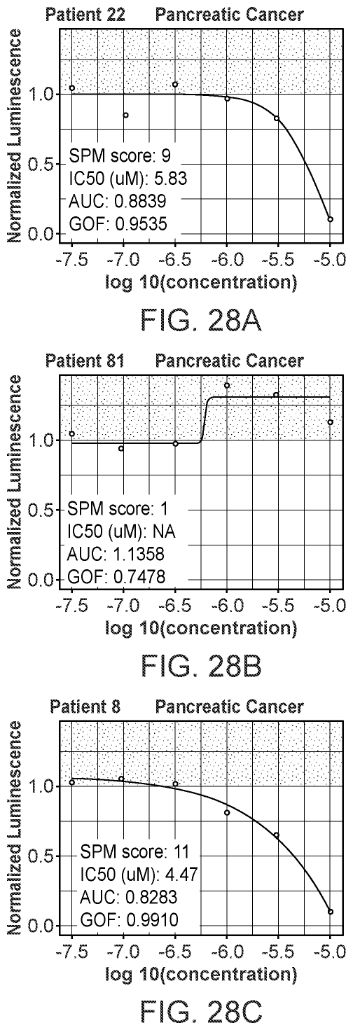

This example describes a pancreatic cancer response to endoxifen measured in patient-derived tumor organoids (PDTOs). Screening of PDTOs was performed as described in EXAMPLE 1. PDTOs derived from three different pancreatic cancer patients were screened for a response to endoxifen. Individual dose-response curves for each patient are shown in FIG. 28A, FIG. 28B, and FIG. 28C. Curve metrics such as the area under the curve (AUC) and goodness of fit (GOF) are provided for each dose-response curve. The IC₅₀ (μM) was provided when it could be calculated by a four-parameter logistic model. If the IC₅₀ was not contained within the range of the tested drug concentrations, the IC₅₀ was not extrapolated. The diagnosis of each pancreatic cancer patient, along with the sensitivity, personalization, and response categories of the PDTO endoxifen response, are summarized in TABLE 9. Additional details for each patient sample are provided in TABLE 1 and EXAMPLE 2.

TABLE 9
Summary of Pancreatic Cancer Screening Results

| | | | Personal- | |
|---|---|---|---|---|
| | | Sensitivity | ization | Response |
| ID | Diagnosis | Category | Category | Category |
| 8 | Pancreatic Cancer | Low | Good | Moderate |
| 22 | Metastatic Pancreatic Ductal | Low | Low | Low |
| | Adenocarcinoma, Stage III | | | |
| 81 | Pancreatic Ductal | None | None | None |
| | Adenocarcinoma | | | |

One pancreatic cancer patient sample was categorized as having a moderate response to endoxifen, and one sample was categorized as having a low response. Together, these data suggest that endoxifen may be an effective treatment for pancreatic cancer.

### Example 13

### Rectal Cancer Response to Endoxifen in Patient-Derived Samples

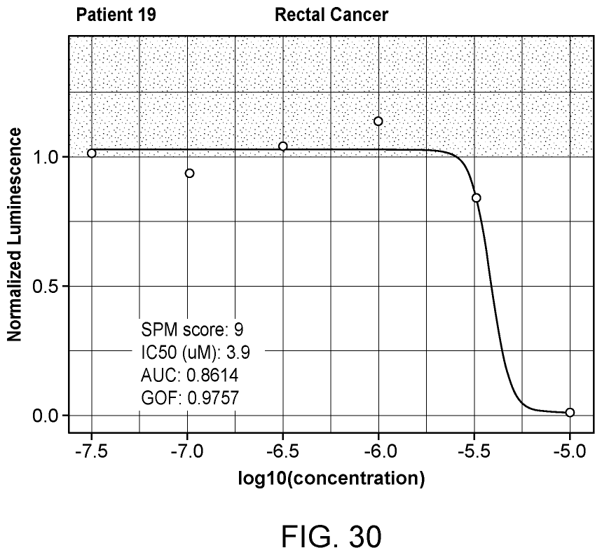

This example describes a rectal cancer response to endoxifen measured in patient-derived tumor organoids (PDTOs). Screening of PDTOs was performed as described in EXAMPLE 1. PDTOs derived from a rectal cancer patient (Patient 19) were screened for a response to endoxifen. The dose-response curve is shown in FIG. 30. Curve metrics such as the area under the curve (AUC) and goodness of fit (GOF) are provided. The IC₅₀ (μM) was calculated by a four-parameter logistic model. The patient had been diagnosed with rectum adenocarcinoma. The sensitivity of the endoxifen response was categorized as low, the personalization of the response was categorized as low, and the response was categorized as low. Additional details the patient sample are provided in TABLE 1 and EXAMPLE 2. This data suggests that endoxifen may be an effective treatment for rectal cancer.

### Example 14

### Treatment of Triple Negative Breast Cancer Using Endoxifen

This example describes treatment of triple negative breast cancer using endoxifen. A patient diagnosed with triple negative breast cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the triple negative breast cancer. Administration of the Z-endoxifen treats the triple negative breast cancer in the patient.

### Example 15

### Treatment of Cervical Cancer Using Endoxifen

This example describes treatment of cervical cancer using endoxifen. A patient diagnosed with cervical cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the cervical cancer. Administration of the Z-endoxifen treats the cervical cancer in the patient.

### Example 16

### Treatment of Cholangiocarcinoma Using Endoxifen

This example describes treatment of cholangiocarcinoma using endoxifen. A patient diagnosed with cholangiocarcinoma is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the cholangiocarcinoma. Administration of the Z-endoxifen treats the cholangiocarcinoma in the patient.

### Example 17

### Treatment of Colorectal Cancer Using Endoxifen

This example describes treatment of colorectal cancer using endoxifen. A patient diagnosed with colorectal cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the colorectal cancer. Administration of the Z-endoxifen treats the colorectal cancer in the patient.

### Example 18

### Treatment of Esophageal Cancer Using Endoxifen

This example describes treatment of esophageal cancer using endoxifen. A patient diagnosed with esophageal cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the esophageal cancer. Administration of the Z-endoxifen treats the esophageal cancer in the patient.

### Example 19

### Treatment of Gastric Cancer Using Endoxifen

This example describes treatment of gastric cancer using endoxifen. A patient diagnosed with gastric cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the gastric cancer. Administration of the Z-endoxifen treats the gastric cancer in the patient.

### Example 20

### Treatment of Melanoma Using Endoxifen

This example describes treatment of melanoma using endoxifen. A patient diagnosed with melanoma is administered Z-endoxifen. The Z-endoxifen is formulated for topical delivery. The Z-endoxifen is administered topically once daily for at least 28 days, until the tumor shrinks, until metastasis slows, or until unacceptable toxicity is observed. Optionally, an additional anti-cancer therapy is used in combination with the endoxifen to treat the melanoma. Administration of the Z-endoxifen treats the melanoma in the patient.

### Example 21

### Treatment of Neuroblastoma Using Endoxifen

This example describes treatment of neuroblastoma using endoxifen. A patient diagnosed with neuroblastoma is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the neuroblastoma. Administration of the Z-endoxifen treats the neuroblastoma in the patient.

### Example 22

### Treatment of Ovarian Cancer Using Endoxifen

This example describes treatment of ovarian cancer using endoxifen. A patient diagnosed with ovarian cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the ovarian cancer. Administration of the Z-endoxifen treats the ovarian cancer in the patient.

### Example 23

### Treatment of Pancreatic Cancer Using Endoxifen

This example describes treatment of pancreatic cancer using endoxifen. A patient diagnosed with pancreatic cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the pancreatic cancer. Administration of the Z-endoxifen treats the pancreatic cancer in the patient.

### Example 24

### Treatment of Rectal Cancer Using Endoxifen

This example describes treatment of rectal cancer using endoxifen. A patient diagnosed with rectal cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the rectal cancer. Administration of the Z-endoxifen treats the rectal cancer in the patient.

### Example 25

### Treatment of Gastrointestinal Adenocarcinoma Using Endoxifen

This example describes treatment of gastrointestinal adenocarcinoma using endoxifen. A patient diagnosed with gastrointestinal adenocarcinoma cancer is administered Z-endoxifen. The Z-endoxifen is formulated for oral delivery. The Z-endoxifen is administered orally once daily for at least 28 days, until the tumor shrinks, until metastasis slows, or until unacceptable toxicity is observed. Optionally, an additional anti-cancer therapy is used in combination with the endoxifen to treat the gastrointestinal adenocarcinoma. Administration of the Z-endoxifen treats the gastrointestinal adenocarcinoma.

### Example 26

### Treatment of Metastatic Skin Cancer Using Endoxifen

This example describes treatment of a metastatic skin cancer using endoxifen. An incidence of skin cancer is identified as being metastatic. The subject with skin cancer is orally administered Z-endoxifen once daily for at least 28 days, until metastasis slows, as measured by diminished cell-free DNA (cfDNA) and circulating tumor cell counts in the subject. Optionally, an additional anti-cancer therapy is used in combination with the endoxifen to treat the metastatic skin cancer. Administration of the Z-endoxifen treats the metastatic skin cancer in the subject.

### Example 27

### Treatment of Metastatic Gastrointestinal Cancer Using Endoxifen

This example describes treatment of a metastatic gastrointestinal cancer using endoxifen. An incidence of gastrointestinal cancer is identified as being metastatic. The subject with gastrointestinal cancer is orally administered Z-endoxifen once daily for at least 28 days, until metastasis slows, as measured by decreases in tumor marker levels through urinalysis. Optionally, an additional anti-cancer therapy is used in combination with the endoxifen to treat the metastatic gastrointestinal cancer. Administration of the Z-endoxifen treats the metastatic gastrointestinal cancer in the subject.

### Example 28

Dual Treatment of Gastrointestinal Cancer with Endoxifen and Radiation Therapy

This example describes treatment of a gastrointestinal cancer using endoxifen and a radiation therapy. A subject is diagnosed with a gastrointestinal cancer. Sites with high incidence of the gastrointestinal cancer are subjected to radiation multiple times per month to achieve a total cumulative radiation dosage of 1 to 100 Gy. The subject is simultaneously orally administered Z-endoxifen once daily for the duration of the radiation therapy, and optionally after conclusion of the radiation therapy. Optionally, an additional anti-cancer therapy is used in combination with the endoxifen to treat the gastrointestinal cancer. The combined radiotherapy, Z-endoxifen course treats the gastrointestinal cancer in the subject.

### Example 29

### Endoxifen Treatment Following Surgical Melanocytic Tumor Removal

This example describes endoxifen treatment following surgical tumor removal. A melanocytic tumor is surgically removed from a melanoma patient. The patient is then administered endoxifen to ensure clearance of remaining cancer cells. The subject is orally administered Z-endoxifen once daily until their melanoma becomes undetectable or until unacceptable toxicity is observed. Optionally, an additional anti-cancer therapy is used in combination with the endoxifen to treat the melanoma. Administration of the Z-endoxifen treats the melanoma in the patient.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a gastrointestinal cancer in a subject in need thereof, the method comprising administering to the subject a therapeutically effective amount of endoxifen, thereby treating the gastrointestinal cancer.

2. The method of claim 1, wherein the gastrointestinal cancer is a colorectal cancer, a gastric cancer, a pancreatic cancer, an esophageal cancer, a rectal cancer, a biliary cancer, a cholangiocarcinoma, or a combination thereof.

3. The method of claim 1 or claim 2, wherein the gastrointestinal cancer is a carcinoma.

4. The method of any one of claims 1-3, wherein the gastrointestinal cancer is an adenocarcinoma.

5. The method of any one of claims 1-4, wherein the gastrointestinal cancer is a colorectal cancer.

6. The method of any one of claims 1-5, wherein the gastrointestinal cancer is a colon cancer.

7. The method of claim 6, wherein the colon cancer is sigmoid colon cancer, adenocarcinoma of the colon, stage III colorectal cancer, stage IV colorectal cancer, or descending colon cancer.

8. The method of any one of claims 2-7, wherein the colorectal cancer is colon cancer, sigmoid colon cancer, adenocarcinoma of the colon, stage III colorectal cancer, stage IV colorectal cancer, or descending colon cancer.

9. The method of any one of claims 1-4, wherein the gastrointestinal cancer is a gastric cancer.

10. The method of any one of claims 1-4, wherein the gastrointestinal cancer is a pancreatic cancer.

11. The method of claim 10, wherein the pancreatic cancer is pancreatic ductal adenocarcinoma, metastatic pancreatic ductal adenocarcinoma, or stage III metastatic pancreatic ductal adenocarcinoma.

12. The method of any one of claims 1-4, wherein the gastrointestinal cancer is an esophageal cancer.

13. The method of claim 12, wherein the esophageal cancer is esophageal adenocarcinoma or metastatic esophageal cancer.

14. The method of any one of claims 1-4, wherein the gastrointestinal cancer is a rectal cancer.

15. The method of claim 14, wherein the rectal cancer is rectum adenocarcinoma.

16. The method of any one of claims 1-4, wherein the gastrointestinal cancer is a cholangiocarcinoma.

17. The method of claim 16, wherein the cholangiocarcinoma is metastatic cholangiocarcinoma.

18. The method of any one of claims 1-17, wherein the gastrointestinal cancer is metastatic.

19. The method of any one of claims 1-18, wherein the gastrointestinal cancer is recurrent.

20. A method of treating a skin cancer in a subject in need thereof, the method comprising administering to the subject a therapeutically effective amount of endoxifen, thereby treating the skin cancer.

21. The method of claim 20, wherein the skin cancer is a melanoma.

22. The method of claim 21, wherein the melanoma is acral melanoma, recurrent acral melanoma, or anorectal melanoma.

23. The method of any one of claims 20-22, wherein the skin cancer is metastatic.

24. The method of any one of claims 20-23, wherein the skin cancer is recurrent.

25. A method of treating neuroblastoma in a subject in need thereof, the method comprising administering to the subject a therapeutically effective amount of endoxifen, thereby treating the neuroblastoma.

26. The method of claim 25, wherein the neuroblastoma is metastatic.

27. The method of claim 25 or claim 26, wherein the neuroblastoma is recurrent.

28. The method of any one of claims 1-27, wherein the endoxifen is (Z)-endoxifen.

29. The method of claim 28, wherein the (Z)-endoxifen has an isomeric purity of at least 90%.

30. The method of any one of claims 1-29, wherein the endoxifen administered orally, topically, rectally, intravenously, intra-arterially, parenterally, transdermally, or via inhalation.

31. The method of any one of claims 1-30, wherein the endoxifen is administered orally.

32. The method of claim 31, wherein the endoxifen is formulated as a sustained-release composition or a delayed release composition.

33. The method of claim 31 or claim 32, wherein the endoxifen is formulated as a capsule or a tablet.

34. The method of any one of claims 1-30, wherein the endoxifen is administered topically or transdermally.

35. The method of claim 34, wherein the endoxifen is formulated is a cream, a gel, a cream, an emulsion, a lotion, an ointment, a solution, a paste, a patch, or an oil.

36. The method of any one of claims 1-35, comprising administering the endoxifen to the subject daily.

37. The method of any one of claims 1-36, comprising administering the endoxifen to the subject at a dose of no less than 1 mg and no more than 160 mg per day.

38. The method of any one of claims 1-37, comprising administering the endoxifen to the subject at a dose of no less than 1 mg and no more than 40 mg per day.

39. The method of any one of claims 1-38, comprising administering the endoxifen to the subject at a dose of no less than 1 mg and no more than 10 mg per day.

40. The method of any one of claims 1-39, comprising administering the endoxifen to the subject at a dose of no less than 2 mg and no more than 5 mg per day.

41. The method of any one of claims 1-40, comprising administering the endoxifen 1, 2, 3, or 4 times per day.

42. The method of any one of claims 1-41, comprising administering the endoxifen for at least 7, at least 14, at least 21, or at least 28 days.

43. The method of any one of claims 1-42, wherein the administering achieves a systemic endoxifen Cm, of between about 0.01 and about 50 μM in the subject.

44. The method of any one of claims 1-43, wherein the administering achieves a systemic endoxifen Cm, of between about 1 and about 10 μM in the subject.

45. The method of any one of claims 1-44, wherein the administering maintains a systemic endoxifen concentration of at least 1 μM for at least one week in the subject.

46. The method of any one of claims 1-45, further comprising administering an additional therapeutic agent.

47. The method of claim 46, wherein the additional therapeutic agent comprises an anti-cancer agent.

48. The method of claim 47, wherein the anti-cancer agent is selected from the group consisting of bicalutamide, enzalutamide, trastuzumab, atezolizumab, alpelisib, olaparib, talazoparib, ribociclib, neratinib, an antineoplastic, capecitabine, carboplatin, cisplatin, cyclophosphamide, docetaxel, doxorubicin, pegylated liposomal doxorubicin, epirubicin, fluorouracil, gemcitabine, methotrexate, paclitaxel, protein-bound paclitaxel, vinorelbine, eribulin, ixabepilone, an immune checkpoint inhibitor, a PD1 inhibitor, a PD-L1 inhibitor, a CTLA4 inhibitor, and an ATP-cassette binding protein inhibitor.

49. The method of claim 46, wherein the additional therapeutic agent comprises a selective serotonin reuptake inhibitor.

50. The method of claim 49, wherein the selective serotonin reuptake inhibitor comprises citalopram, escitalopram, fluoxetine, paroxetine, sertraline, or vilazodone.

51. The method of any one of claims 1-50, further comprising surgical tumor removal.

52. The method of claim 51, wherein the administering the therapeutically effective amount of endoxifen is concurrent with, subsequent to, or concurrent with and subsequent to the surgical tumor removal.

53. The method of any one of claims 1-52, further comprising radiotherapy.

54. The method of claim 53, wherein the administering the therapeutically effective amount of endoxifen is concurrent with, subsequent to, or concurrent with and subsequent to the radiotherapy.
